(19) United States
(12) Patent Application Publication
(10) Pub. No.: US 2026/0083824 A1
(43) Pub. Date: Mar. 26, 2026

(54) ENRICHMENT AND CHANNELING OF PLURIPOTENT CELLS BY PROTEIN NANOPARTICLES

(71) Applicant: Richard C.K. Yen, Yorba Linda, CA (US)

(72) Inventor: Richard C.K. Yen, Yorba Linda, CA (US)

(21) Appl. No.: 19/410,002

(22) Filed: Dec. 5, 2025

Related U.S. Application Data
(63) Continuation-in-part of application No. 19/190,246, filed on Apr. 25, 2025.
(63) Continuation-in-part of application No. 18/812,320, filed on Aug. 22, 2024.
(63) Continuation-in-part of application No. 18/735,523, filed on Jun. 6, 2024.
(63) Continuation-in-part of application No. 18/295,829, filed on Apr. 4, 2023.
(60) Provisional application No. 63/100,670, filed on Mar. 25, 2020.
(60) Provisional application No. 63/372,883, filed on Apr. 13, 2022.
(60) Provisional application No. 63/628,513, filed on Jul. 31, 2023.
(60) Provisional application No. 63/630,952, filed on Mar. 18, 2024.
(60) Provisional application No. 63/833,529, filed on Dec. 12, 2024.
(60) Provisional application No. 63/834,098, filed on Feb. 21, 2025.

(51) Int. Cl. A61K 38/38; A61K 38/36

(52) U.S. Cl. CPC A61K 38/38; A61K 38/363

(57) ABSTRACT

Fibrinogen-coated Albumin Nanospheres (FAS) has shown that it can mobilize bone marrow cells into the blood which then go to injured soft and hard tissues to achieve accelerated repair. It is hypothesize that (a) brain injuries caused by low-dose irradiation under simulated microgravity can be mitigated by the administration of FAS; and (b) the mechanism is from acceleration of neo-vascularization, proven by lineage study of GFP-labeled bone marrow cells implanted into compatible non-GFP animals before injury, which regenerate into functional GFP-brain cells after FAS treatment.

### CROSS-REFERENCE TO RELATED APPLICATION

This application claims the benefit of priority under 35 U.S.C. § 119 (e) based upon co-pending U.S. provisional patent application Ser. No. 63/833,529 filed on Dec. 9, 2024, and co-pending U.S. provisional patent application Ser. No. 63/834,098 filed on Feb. 18, 2025. The entire disclosure of the prior provisional application is incorporated herein by reference.

This application is a continuation-in-part under 35 U.S.C. § 120 based upon co-pending U.S. patent application Ser. No. 18/812,320 filed on Aug. 22, 2024, co-pending U.S. patent application Ser. No. 18/295,829 filed on Apr. 4, 2023, co-pending U.S. patent application Ser. No. 18/735,523 filed on Jun. 6, 2024, and co-pending U.S. patent application Ser. No. 19/190,246 filed on Apr. 25, 2025.

The entire disclosure of the prior provisional applications are incorporated herein by reference.

### BACKGROUND

### Technical Field

In some aspects, the present technology relates to an enrichment and channeling of pluripotent cells by protein nanoparticles for use in connection with regenerative biology and medicine. In some other aspects, the present technology relates to protein nanoparticles and methods associated with protein nanoparticles to enrich and channel (preferential differentiation of) pluripotent cells including but not limited to those harvested from the bone marrow, muscle and adipose tissues. In other aspects, the present technology relates to the use of protein nanoparticles which can be administered intravenously to achieve such medically beneficial effects. In yet other aspects, the present technology relates to the use of protein nanoparticles as separation agents in vitro, with the result that certain pluripotent cells and their progeny cells become enriched in their concentration or function, leading to unexpected medical benefits.

### Background Description

Pluripotent cells are cells that have the capacity to differentiate (from their “primitive state”) into mature functional cells which form the tissues and organs of the body. They are often called stem cells; and historically stem cells are recognized as being located inside the bone marrow. These stem cells develop into various “lineages” such as the “hematopoietic lineage” which are the differentiated cells which form the various cellular components of the blood. These cells have distinct cellular morphology recognizable by histologists to be the various types of cells such as red blood cells, white blood cells and platelets; and their subtypes. However, recent research revealed that some “stem cells” can be located in other tissues, such as the endothelium—these cells are known to differentiate also, e.g. into bone tissues; and therefore, these cells are often called “progenitor cells.” In addition, some cells can be flushed from various organs (e.g. kidneys) which are called “perivascular stromal cells” which can also be useful as regenerative cells (“Clinical-Grade Isolated Human Kidney Perivascular Stromal Cells as an Organotypic Cell Source for Kidney Regenerative Medicine” authored by Leuning et al, at Stem Cells Transl Med. 2017 February; 6(2):405-418.) In addition, pluripotent cells can be isolated from many other tissues, such as the muscle or the adipose tissues. Therefore, regardless of their different location of origin, in this disclosure all these “regenerative” cells as a group will be called pluripotent cells (even though some may be totipotent), or stem cells or progenitor cells.

For patients who need stem cell therapy, there are two main methods of getting these regenerative cells:
a) by harvesting the patient's own stem cells or by induction of some mature cells obtained from the patient (i.e. conversion of certain mature cells into less mature cells which can then differentiate into the “wanted” cell types; and
b) by the administration of cells obtained from sources other than the patient himself, e.g. umbilical cord cells or bone marrow cells obtained from a compatible donor.

By definition, all these harvested (primitive) cells need to undergo certain “processes” in vitro (therefore, they are to be called “processed cells” in this disclosure) to become more useful for medical indications; e.g. (a) by harvesting the stem cells and then directly enriching their concentration in vitro, or (b) by an indirect approach, e.g. first by inducing certain mature cells harvested from the patient into (less mature) pluripotent cells (“initial processed cells”), and then further inducing these “initial processed cells” further towards a particular pathway (“desired processed cells”, the purpose being to avoid formation of “unwanted” cells. Unwanted cell can occur if the “initial processed cells” were used for implantation, which can develop into tumor cells. These steps are complicated and expensive, not to mention that there is no guarantee that the “desired processed cells” will perform well without side effects.

One common problem of using various methods to obtain “processed cells” is that there is no assurance that only the “wanted” differentiated cells will be formed. First, the (harvested) contributing cell population may have a wide range of cells, some of which are already committed to different pathways or lineages, even though they all look similar (being all “primitive-looking” in their cellular characteristics.) Second, the processing method may not be able to stimulate the cells toward only one particular pathway, or any pathway at all. Third, the processed cell population may not be compatible with the patient after they are infused/transfused into the patient. Even if the original cells were harvested as mature cells from the patient (to be reprogrammed into less mature cells) there is no assurance that the various steps of the long process would not have changed the characteristics of the cells, making them problematic for the recipient.

At the most fundamental level, for patients who need total bone marrow transplant, they will first have to undergo ablation (i.e. destruction of all of the patient's own stem cells) and then the donor cells will be infused (typically intravenously) to achieve the transplant. After the infusion of the “best possible” donor bone marrow cells, the fate is either “take” or “not take.” There is at the present time no technology available to modify the maturation/differentiation of the transplanted stem cells, for the benefit of the patient. In addition, a total-ablation would have killed a large number of stem cells which are needed to sustain the on-going well-being of the patient. These patients are often susceptible to infections for lack of an adequate concentration of white blood cells. Therefore, recently the approach has been changed to “partial-ablation” of the host before infusion of the donor cells.

One fear of clinicians advising “partial-ablation” to cancer patients is the possibility that the lower dose of chemotherapy or radiation would result in a higher concentration of cancer cells being able to survive the pre-treatment. However, to everybody's surprise, the infused (donor) cells often retain their immune ability and could in a beneficial way be used to kill the remaining cancer cells left in the host: this being the benefit of a “guest” killing off the unwanted tenants which the owner of the house had been trying to evict for a long time (without success).

Therefore, there is a need for more advanced technologies which can (a) enrich the pluripotent cells including bone marrow cells or cells from other tissues, so that a greater assurance can be obtained that the desirable “end product” (i.e. functional cells at the target tissue) can be generated; and (b) promote the development of the lineages of cells needed at the target level of the patient (e.g. the wound site or a site that needs rejuvenation.)

One goal of the present technology is to disclose a new technology by the use of nanoparticles, which can achieve the above goals so that (1) bone marrow transplant patients can benefit; and (2) from the knowledge gained here, the non-bone-marrow-transplant patients who have other medical problems that can benefit from some invigoration of their own bone marrow cells (endogenous and not transplanted) or from implantation of other pluripotent cells (obtained less-invasively than bone marrow harvesting), that they too can achieve a greater degree or faster degree of healing.

### SUMMARY

In view of the foregoing disadvantages inherent in the known types of methods to obtain “processed cells” at least some embodiments of the present technology provides a novel enrichment and channeling of pluripotent cells by protein nanoparticles, and overcomes one or more of the mentioned disadvantages and drawbacks of the prior art. As such, the general purpose of at least some embodiments of the present technology, which will be described subsequently in greater detail, is to provide a new and novel enrichment and channeling of pluripotent cells by protein nanoparticles which has all the advantages of the prior art mentioned herein and many novel features that result in an enrichment and channeling of pluripotent cells by protein nanoparticles which is not anticipated, rendered obvious, suggested, or even implied by the prior art, either alone or in any combination thereof.

According to one aspect, the present technology can include a method of using an albumin nanoparticle suspension containing submicron albumin spheres to enrich and channel pluripotent cells in a subject in need thereof. The method can include the step of administering intravenously a therapeutically effective amount of the albumin nanoparticle suspension containing the submicron albumin spheres to the subject. The albumin spheres can be configured to provide acceleration of in vivo neo-vascularization of the pluripotent cells to enrich one or more of the pluripotent cells into desirable cells for proper physiological function and to channel the one or more enriched pluripotent cells so the enriched pluripotent cells are reprogrammed to differentiate into pathways useful to the subject.

According to another aspect, the present technology can include a method of using an albumin nanoparticle suspension containing submicron albumin spheres to enrich and channel pluripotent cells in a subject in need thereof. The method can include the steps of providing a suspension including fibrinogen-coated albumin nanospheres prepared by coating blank albumin spheres with a solution containing human fibrinogen. Administering intravenously a therapeutically effective amount of the albumin nanoparticle suspension to the subject. Enriching one or more of the pluripotent cells into desirable cells for proper physiological function by in vivo neo-vascularization of the one or more pluripotent cells. Channeling the one or more enriched pluripotent cells so the enriched pluripotent cells are reprogrammed to differentiate into pathways useful to the subject.

In some embodiments, the albumin spheres of the albumin nanoparticle suspension can be bound with fibrinogen molecules to produce Fibrinogen Albumin Spheres (FAS).

Some embodiments of the present technology can include a step of increasing a concentration of CD34+ or CD31− cells by the Fibrinogen Albumin Spheres.

In some embodiments, the channeling of the enriched pluripotent cells can be configured to replenish a depletion of stem cells in bone marrow of the subject.

Some embodiments of the present technology can include the steps of:
moving of the FAS from an intravenous compartment into a bone marrow compartment of the subject;
attachment of the FAS to the pluripotent cells, and signaling to the pluripotent cells to reproduce to create FAS-attached pluripotent cells;
exiting of the FAS-attached pluripotent cells from the bone marrow;
maturation of the FAS-attached pluripotent cells in peripheral blood of the subject;
arriving of the FAS-attached pluripotent cells at a target site of the subject; and
healing macroscopically or histologically at the target site by an abundance of the FAS-attached pluripotent cells at the target site.

In some embodiments, the depletion of stem cells can be caused by ablation or partial ablation.

In some embodiments, the desired cells can be functional cells at a target tissue of the subject.

The method according to claim 1 further comprising the step of promoting by the albumin spheres a development of lineages of pluripotent cells needed at a target tissue of the subject.

In some embodiments, the lineages can be of hematopoietic cells.

In some embodiments, the pluripotent cells can be from a donor that is not the subject.

Some embodiments of the present technology can include a step of increasing an effectiveness of engrafting of the donor pluripotent cells in bone marrow of the subject resulting in an increase in survivability of the donor pluripotent cells in the bone marrow of the subject and to become mature cells later.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel enrichment and channeling of pluripotent cells by protein nanoparticles that has all of the advantages of the prior art methods to obtain “processed cells” and none of the disadvantages.

It is another object of the present technology to provide a new and novel enrichment and channeling of pluripotent cells by protein nanoparticles that may be easily and efficiently manufactured and marketed.

An even further object of the present technology is to provide a new and novel enrichment and channeling of pluripotent cells by protein nanoparticles that has a low cost of manufacture with regard to both materials and labor, and which accordingly is then susceptible of low prices of sale to the consuming public, thereby making such enrichment and channeling of pluripotent cells by protein nanoparticles economically available to the buying public.

Still another object of the present technology is to provide a new enrichment and channeling of pluripotent cells by protein nanoparticles that provides in the apparatuses and methods of the prior art some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a reproduction of FIG. 2 of the prior art article by Daneil.

### DETAILED DESCRIPTION OF THE EMBODIMENTS

While the above-described devices fulfill their respective, particular objectives and requirements, the aforementioned devices or systems do not describe an enrichment and channeling of pluripotent cells by protein nanoparticles that allows enriching and channeling of pluripotent cells. The present technology additionally overcomes one or more of the disadvantages associated with the prior art.

A need exists for a new and novel enrichment and channeling of pluripotent cells by protein nanoparticles that can be used for enriching and channeling of pluripotent cells. In this regard, the present technology substantially fulfills this need. In this respect, the enrichment and channeling of pluripotent cells by protein nanoparticles according to the present technology substantially departs from the conventional concepts and designs of the prior art, and in doing so provides an apparatus primarily developed for the purpose of enriching and channeling of pluripotent cells.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

One of the goals of the present technology is the increase in the concentration or the total mass of useful cells in a patient so that the wounds, deficiencies, or dysfunctions (genetic or non-genetic) of tissues and organs can be mitigated or repaired. The term “useful cells” will refer to any number of cells in between the initial pluripotent cell that will regenerate into functional cells in a wounded or defective organ, to the cells that have arrived at the end organ or tissue, and have become the normal part of the histological organization of the tissue or organ.

One example of a patient population would be cancer patients who need bone marrow transplantation. At the present time, these patients are mainly blood-cell cancer patients, e.g. those suffering from leukemia. The patients are treated first with “ablation” which means the use of chemotherapy or radiation to kill off most of the cancer cells which reside in the bone marrow (or elsewhere in the body), which also kill off much of the normal regenerative cells in the bone marrow. Then donor bone marrow cells (which are pluripotent cells) are harvested from compatible donors and transfused into the ablated patient so that the donor cells can move into the bone marrow to replace the cells (normal as well as cancerous) previously residing in the bone marrow. Without the proper implantation of the donor cells into the bone marrow and their subsequent differentiation into all the blood-cells needed for life, the cancer patient will be at risk of death (from the ablation if not from the residual cells of the cancer.) This type of treatment is most often used to treat blood cancers because these cancer cells are most easily killed by ablation and the transplanted bone marrow cells are efficient in the replenishment of (non-cancer) blood cells. Unfortunately, not all cases of transplantation will result in the donor cells being able to function, i.e. the success rate of bone marrow transplant is not 100%; and there are other complications, such as graft-vs-host diseases. The present technology is designed to increase the success rate of transplantation as well as a decrease in the rate of complications.

At the present time, because of cost and other associated risks, there is not much done using this “bone marrow transplant” method to treat non-hematopoietic cancer cases, or non-cancer patients including patients with large or difficult-to-treat wounds. However, there are other issues where the cells have high turn-over, meaning a large number of these cells get old fast and die fast and they need to be replaced by new and functional cells of the same kind, such as cells in the gastrointestinal tract and the skin. The present technology will, therefore, be able to benefit non-cancer patients, in addition to cancer patients (whether the cancer is related to the blood or related to non-blood tissues.) Since the bone marrow cells (from the patient or from the donor) can differentiate into many mature cell types—that is why bone marrow cells are called pluripotent cells—the key again is to enrich the particular stem cell lines so that the needed cell types are increased in concentration or in effectiveness to provide the patient with the proper physiological function, in place of the previously cancerous or non-cancerous but dysfunctional cells, or to mitigate other deficiencies or dysfunctions in the damaged or injured tissues or organs.

The present technology is also aimed at “channeling” of stem cells or pluripotent cells so that they are “encouraged” or induced, or selected, to become mainly the type of cells beneficial to the patient and that these “pluripotent cells” do not differentiate into cells that are not desirable for the need of the patient. The term “channel” or “channeling” in this disclosure can mean (a) of the many possible differentiation pathways, each represented by a lineage from certain stem cells, the present technology would reprogram some of them so that they do not differentiate into “meaningless” pathways (ending in cell types not useful to the patient) but would mainly differentiate into the meaningful pathways useful to the patient; and/or (b) the selection of certain stem cells (in vivo or in vitro) among many different (probably committed) stem cells so that there is an increased chance of the wanted stem cells (leading to the wanted end-products) being used to implant the bone marrow (or other locations) of a patient, leading to higher rates of clinical success and lower rates of complications. In other words, regardless of the mechanism of action, the end-result by the use of the present technology will be an increased number of useful cells compared to situations where the present technology is not used or not available.

Another example of the application of the present technology is in non-cancer patients, e.g. patients suffering from severe or hard-to-heal wounds, including multiple-trauma such as gunshot wound, burn, brunt trauma, infected wounds, dirty bomb (contaminated with radioactive material.) Since these patients do not receive bone marrow ablation, they have a native (endogenous) population of bone marrow cells. In fact, recent discoveries have shown that there are stem cells outside the bone marrow, such as in muscles and adipose tissues. Therefore, in this disclosure the term “stem cell” can mean pluripotent or totipotent cells located in anywhere in the body. We expect these non-cancerous patients to also benefit from the present technology.

The present technology envisions multiple benefits to the patient, by the enrichment of useful cells of different origins (i.e. originating from the patient, or from the donor) and cells from different locations (those residing in the bone marrow or outside the bone marrow.) The benefit is related to the enrichment of useful cells or their preferential selection by use of protein nanoparticles or the ability of the nanoparticles to reprogram cells into useful cells.
(I) Regarding the effect of protein nanoparticles administered to healthy animals (no bone marrow transplant or any other cellular infusions from outside sources), Yen has disclosed data showing the effect of protein nanoparticles on the bone marrow and other tissues as described below.

Yen disclosed that certain protein nanoparticles or nanospheres can be administered via the intravenous route, which will result in the attachment of such nanoparticles to the cells residing in the endothelium of the blood vessels. In addition, these nanospheres (called “Fibrinogen-coated albumin spheres, FAS) can also attach to bone marrow cells inside the bone marrow. Furthermore, administration of FAS intravenously has been shown to increase CD34+ (stem) cells inside the bone marrow, followed by increased concentration of certain “more mature” cells (e.g. lymphocytes, monocytes, granulocytes) in the peripheral blood. The prior disclosures include a non-provisional patent application titled “Protein Nanospheres to Treat Harm from Multiple Trauma.”

Yen has also disclosed the use of the “biological product” called Fibrinoplate-S (FPS) which is a suspension of the “biological substance” (or active substance) called Fibrinogen-coated albumin spheres (FAS). Several separate experiments (to be described in the following paragraph) using different animal models suggest that the intravenous administration of FPS can lead to the mobilization of stem cells whether these stem cells originate from the bone marrow or from endothelial cells or from other locations. In addition to the macroscopic observation that the FPS- or FAS-treated group had accelerated healing of the wounds compared to the control group, CD34+ stem cells could still be detected in the biopsies of the wound (or healed-wound-area) and these CD34+ cells are in far greater abundance in the FAS-group than in the saline-group. This is particularly interesting because if the wound had healed, those stem cells that brought about the healing are supposed to have differentiated into mature cells such as muscles, nerves, blood vessels, fat, skin, depending on where they have “landed”—to achieve healing of the many kinds of tissues which had been previously injured. During differentiation, the stem cells that have arrived at the wound should all have shed their original CD34+ cell marker (because the differentiated and functional cells typically will have other cell markers, but not the CD34 marker.) The fact that CD34+ cells are still observed In the healing (or healed) wound biopsy, far more in the FPS- or FAS-group than the saline-group, and at many weeks after one dose of FPS administration, suggests strongly that there must be an overabundance of CD34+ stem cells having arrived at the wound at the various stages of healing, many of which still retain their “primitive” CD34+ cell marker, even at the time of biopsy (e.g. on day 51 after FPS administration) when the wound is observed to have almost completely healed.

However, it should be noted that Yen had not achieved or even described a demonstration that these separate events are linked with each other longitudinally, i.e. one event gives rise to the next event in the same animal. Yen has not described a “lineage” study, linking the initial event (attachment of FAS to bone marrow cells) to the end product/event (healing of a wound.) Also, these published prior-art events are observed so far only in non-transplanted animals. One goal of this disclosure is to show that the events described in the following paragraph are physically linked (one event giving rise to another) when they occur in healthy animals as well as in bone-marrow transplanted animals.

Specifically, regarding the mobilization of stem cells by the administration FPS. In non-marrow-transplanted animals, a dose of FPS administered intravenously has been shown (a) to result in the entry of the active drug substance (FAS) into the bone marrow compartment of the bone marrow of healthy mice. This experiment used FITC (fluorescein isothiocynate)-labeled FAS infused intravenously into healthy mice. Following isolation of bone marrow cells from these mice, FITC-FAS can be seen in flow cytometer to be still attached to the isolated bone marrow cells. (b) In separate experiments, unlabeled (original) FAS administered intravenously to mice has been shown to result in an increase of CD34+ cells in the bone marrow. (c) In separate experiments using mini-pigs, an increased concentration of CD34+ cells can also be detected in the blood soon after administration of FPS. (d) In rats suffering from skin injury induced by a high dose of local irradiation onto the skin causing a visible wound within a month, one dose of FPS given after the appearance of the skin lesion will result in the healing of the wound within 9 days and an abundance of CD34+ cells can still be detected in the biopsy obtained at this late stage. (e) In mini-pigs irradiated at the skin with 50 Gy of irradiation, around 50 days after the administration of FPS, the skin biopsy of the FPS-group showed complete recovery of blood vessels. It is well known that high doses of irradiation can greatly damage blood supply by the destruction of blood vessels. In some areas in the biopsy, the FPS-group showed (by histological staining) normal intensity and distribution of CD31+ cells (which labels endothelial cells): showing that neo-vascularization is essentially complete by that time, giving the skin of the FPS-group a chance to receive normal nutrients from the blood (because the blood vessels are now functional in that area due to the healing effects of the FPS, via the mobilization of stem cells.) In addition, the other tissues (other than blood vessels) such as fat, muscle, nerves have all healed much better in the FAS-group than the control (saline)-group.

Recently, the intravenous administration of FAS has also been shown to accelerate the mineralization of healing bones in vivo after a fracture. At the same time, it was discovered that there are more than twice the number of CD34+ stem cells at the callus site of the FAS-treated group compared to the saline-control group (U.S. Pat. No. 12,161,697, “Nanospheres for Bone Fracture”, issued Dec. 10, 2024.) Therefore, FAS appears to be effective in the healing of soft-tissue wounds such as a skin wound, as well as hard tissue injuries such as bone fractures. The mechanism of healing in all of the above cases is most likely due to the mobilization of CD34+ cells some of which became blood vessels with intact endothelium (staining positive for CD31 cell marker) while others became mature cells such as nerves and muscles, seen in the slides of the biopsies by standard methods of staining.

All of the above data showed that (a) FAS administered intravenously can be beneficial to the healing of a variety of wounds; (b) despite the visible healing of the wound (by macroscopic observation as well as histological studies of the biopsies) which requires differentiation of the (originally) stem cells into the mature tissues at the wound site, there are still enough CD34+ cells leftover at the wound site which have not shed their CD34 cell markers.

However, the above data did not answer the following questions:
1) What are the identity of the bone marrow cells that the FITC-labeled FAS (and non-FITC-labeled FAS) can (or do) attach to and what is the effect on these cells by the attachment of the FAS? In other words, (a) is there a preferred population of cells which the FAS will attach to inside the bone marrow which will give rise to daughter cells that will appear later in the blood or at the wound site? Or (b) is it possible that all potentially useful lineages of cells can be attached by FAS in the bone marrow?
2) Does the attachment of FAS to the bone marrow cells cause or result in some kind of stimulation of the attached bone marrow cells? Afterall the FAS has fibrinogen molecules on their surface and many cells have fibrinogen-receptors. Will the attachment lead to a more concentrated volume of useful cells, or will it also lead to a wider array of benefits to a wide variety of medical indications?
3) Are there other sites outside the bone marrow that FAS can attach to? We already know that FAS can attach to the endothelial cells inside a live animal (by using 2-photon microscopy); but the same question applies—is that a specific population of endothelial cells or all “pluripotent” endothelial progenitor cells on the endothelium can be activated by the attachment of FAS in vivo?
4) Can we track the activated stem/pluripotent cells (from whatever location) to their differentiated states (and localizations of functionality within the body)? i.e. can we do a “lineage” study?
5) Do transplanted bone marrow cells respond the same way as the patient's own endogenous bone marrow cells to the effects of FPS administration in vivo?
6) Can we improve the chances of success for bone marrow transplants by the use of FAS, in vivo?
7) Can we use FITC-FAS as a tool for the separation of useful cells in vitro, to isolate preferred populations of isolated bone marrow cells in vitro (in conjunction with a cell sorter, using flow cytometry)? An alternative method of isolating FITC-FAS-bound bone marrow cells is to use magnetic spheres which have anti-FITC IgG: the magnetic spheres will first be used to bind FITC-labeled-FAS (which have bound to the desired bone marrow cells in vitro) followed by magnetic separation of the magnetic spheres with their attached “load”.
8) Are there potential harmful effects from FPS administration, e.g. the stimulation or induction of blood cancer cells, in either the normal or the transplanted stem cell population, or in the non-stem-cell population (e.g. supportive cells) in the bone marrow or elsewhere? However, as of this time, cGLP (current Good Laboratory Practice) studies in rats have shown no adverse effects in all of the tissues when the rats received intravenous doses of FAS up to 160 mg/kg, nor were any clinical signs observed.
9) At the present time it is not certain whether a healthy person would have only a limited number of (‘regenerative”) stem cells that can give rise to other “equally pluripotent” stem cells, or whether the reservoir of stem cells is really limited. If the reservoir of pluripotent cells is limited in some cases, would the stimulation caused by the infusion of FPS produce a “depletion” of this reservoir, leading to future “early demise” of certain cell lineages, if not the patient himself?
10) Can an improvement on the composition of the FAS, e.g. by decreasing (or increasing) the size of the FAS itself (from around 0.2 micron) help overcome the difficulties of the spheres in attaching to endothelial cells? The endothelial cells are all subject to huge shear forces due to the close proximity (and high velocity) of the fluid (blood) rushing by the endothelium and the constant pounding and pumping force of the cardiovascular system. Therefore, a smaller volume of each of the individual sphere (protruding from the endothelium after the sphere attaches) may help the initial attachment and retention of the sphere on the endothelium? Or an increase of fibrinogen content (or decrease) per FAS sphere may also increase the effectiveness of the FAS? (Note: A method of producing “high fibrinogen spheres” has been submitted in a Provisional patent application sent to the USPTO on Dec. 9, 2024, titled “Protein Nanospheres to Treat Dysfunction from Chemotherapy and Immunosuppressive Therapy.)

In other words, the previous data showed the existence of separate events leading to the end-point of certain medical benefit to the patient, in that the wound caused by even different mechanisms of injury can be healed in terms of faster healing and more complete healing. These separate (and probably intermediate) and eventually-beneficial events can occur at the bone marrow level, at the peripheral blood level and at the wound healing site level. However, the effects of FAS administration deserve more thorough research. (a) Each of these intermediate events can have multiple effects (or divergent directions) not necessarily all linked to the end-point of healing. For example, the FAS may be able to attach to “10” different kinds of bone marrow cells, all of which have different effects, depending on the immediate need of the patient—but Yen had studied only one effect in each of the experiments (with focus on wound healing.) Yen has not studied the “other 9 potentially useful effects of FAS administration.” We have not mapped out all the other potential effects of FAS administration on the bone marrow or other sites affected by FAS. (b) In terms of end-points, it is also not obvious whether the effect of FAS administration leads to only one pathway or whether multiple pathways can exist, so that a patient with multiple wounds or polytrauma can benefit at all levels from the administration of one drug (i.e. FPS, or FAS.)

The work disclosed here is designed to answer some of the above questions. In particular, we intend to discover if various regiments of FAS administration (the dose and the timing of the dose of FAS) can channel the differentiation of bone marrow cells inside the bone marrow so that there will be an enrichment of the desired cell population at the wound site(s). In addition, we wish to find out if the presence and nature of a wound (being a single wound or a multitude of wounds and where they are located in the body) would influence the path of differentiation of the stem cells, with and without the administration of FPS.
(II) Regarding patients that need or are undergoing bone marrow transplant: there is no prior art discussing or disclosing the effect of the administration of protein nanoparticles on the cells in the bone marrow. Typically, there are at least 2 kinds of cells in the bone marrow (before donor transplant) and at least 3 kinds of cells (after donor transplant.) The support cells in the bone marrow are called “stromal cells.” The stem cells that are pluripotent are called “parenchymal cells.” Before the transplant procedure, these two kinds of cells both originate from the patient. Parenchymal cells do at least two things: (a) regenerate themselves into other (additional) pluripotent stem cells and (b) differentiate into more mature cells. For bone marrow transplant, the patient's own bone marrow is typically ablated first to remove the undesirable parenchymal cells (usually the cancer cells). However, the ablation is often incomplete. Then the donor cells are infused, which will enter the patient's bone marrows to reside there. Therefore, after the transplantation event, the patient will have three kinds of cells: his own stromal cells, his own left-over parenchymal cells and the new donated parenchymal cells from the donor. We intend to study (a) the transplanted donor cells, (b) any left-over endogenous parenchymal cells, (c) the stromal cells of the host. We particularly intend to study the effect of an intravenous dose of FAS on all the cells in the bone marrow, whether they are of the patient's own origin or from the donor. We also intend to use FAS as a tool to isolate certain population of bone marrow cells in vitro before their infusion as “preferred donor cells” to repopulate the ablated bone marrow environment.

Yen's disclosure and all prior art had no suggestion on how protein nanoparticles would have affected the grafting of donor bone marrow cells (or stem cells), their maturation inside the bone marrow or their exit from that site; nor any potential effect on what cell types would have been positively enriched (or diminished) and whether they have increased likelihood of differentiation into the mature cells. In addition, there is no indication that any of the prior arts would have predicted whether the administration of protein nanoparticles intravenously into a host which had been bone-marrow-ablated and had received certain kinds of donor cells (whether from self, relatives, or strangers including umbilical cord cells) would have greater (or less) benefits in terms of providing healing in a host who also has a wound or physiological defect that can benefit from accelerated stem cell differentiation into the needed cells to heal the wound or repair a defect. Therefore, this disclosure on the effect of protein nanoparticles on bone marrow cells that have been transplanted into a host is novel and non-obvious; the novelty and non-obviousness apply to both the non-injured hosts (who is without known wounds or defective organs) and the injured hosts (with wounds or dysfunctional organs.)

In this disclosure, when we reveal the positive effect of FAS on the host, we do not rule out the possibility that the benefit comes from the supportive cells in the bone marrow or other “stem cell sites” (including the endothelium or other perivascular sites or in the adipose sites). In addition, the benefit can come from soluble factors generated by any of the cells. In most cases, we expect the benefit of FAS administration to come from the direct effects of FAS on the stem cells.

To show that one cellular event is the cause for a subsequent event, it is vital that daughter cells be traceable. It is now possible to label stem cells by the introduction of “bioluminescent genes” or “fluorescent genes” (i.e. genes incorporated into the animal's DNA, which will make proteins that produce bioluminescence or fluorescence.) For bioluminescent cells, upon the provision of the appropriate substrates, and under the appropriate light source, progenies of these “transgenic” cells (i.e. cells with the bioluminescent genes inserted into their DNA) can reveal in vivo (and in vitro) that they are indeed the descendants of the originally implanted cells and what they have become (or where they have gone to) in the body. Regarding the potential of using induced stem cells (induced from mature cells) to populate the bone marrow of a host, where the donor cells had been “inserted” (transduced) with bioluminescence and fluorescence genes so that their progeny can be traced, there is an excellent article by Vogel et al. The article was titled “The in vivo timeline of differentiation of engrafted human neural progenitor cells” published in Stem Cell Research Vol 37, May 2019, 101429. The Introduction stated: “Understanding the individual timeline of stem cell differentiation in vivo is critical for evaluating stem cell properties in animal models. However, with conventional ex vivo techniques, such as histology, the individual timeline of differentiation is not accessible. Therefore, we designed lentiviral plasmids with cell-specific promoters to control the expression of bioluminescence and fluorescence imaging reporters. Promoter-dependent reporter expression in transduced human induced pluripotent stem cell-derived neural progenitor cells (hNPCs) was an effective indicator of differentiation in cell culture. A 12-week in vivo imaging observation period revealed the time profile of differentiation of engrafted hNPCs in the mouse brain into astrocytes and mature neurons which was verified by immunostainings, patch-clamp electrophysiology, and light-sheet fluorescence microscopy. The lentiviral vectors validated in this study provide an efficient imaging tool box for non-invasive and longitudinal characterization of stem cell differentiation, in vitro screenings, and in vivo studies of cell therapy In animal models.” However, this article did not suggest the possibility of using protein nanospheres or nanoparticles to enhance the grafting process or to see whether other positive effects can be achieved as disclosed in the present technology. Therefore, the administration of FAS to influence the fate of transplanted bone marrow cells which can be traced to their progeny population is a new approach which is both novel and non-obvious.

The use of bioluminescence has several limitations, mainly the need to feed the substrate to the cells within a short time of using an illuminating light source to shine onto the organ or site. If a site is not penetrable (or has low uptake) by the substrate, then the site will not show bioluminescence and thus provides a “false negative” result.

Fluorescent proteins on the other hand provide a more stable source of illumination. A list of such proteins can be obtained from “Fluorescent Proteins for Flow Cytometry” by Hawley et al. published in Curr Protoc Cytom. 2017 Apr. 3; 80:9.12.1-9.12.20. In addition, the genes can be turned on upon initiation of a certain physiological event or can be turned on “at all times.” One example of such fluorescent proteins is the Green Fluorescent Protein (GFP) which produces “green cells” in the bone marrow as well as in the spleen, the thymus and the blood cells. In addition, antibodies against the Green Fluorescent Protein (GFP) or similar proteins (the antibody being linked to enzymes) can greatly enhance the signal from GFP-containing cells by the use of color-generating reactions from the linked enzyme and therefore, does not require the use of fluorescent microscope and other expensive equipment. We intend to use all of the above new technologies to demonstrate the benefits of using protein nanospheres for the enrichment of bone marrow cells or their channeling toward useful cells.

### Planned Experiments and Expected Results

Part One: Effect of FAS on the Pluripotent Cell Population which are Harvested from Various Sites (Bone Marrow and Outside Bone Marrow) on (1) their Grafting Efficiency; (2) Whether a Special Lineage of Stem Cells are Favored and Channeled Later into Useful Mature

### Cells

### Experiment One:

Effect of the timing of the protein nanoparticle administration to a donor, on the success of engraftment and development of transplanted bone marrow cells from the donor to a “total ablation” recipient.

### Introduction:

According to “Types of Stem Cell and Bone Marrow Transplants” (American Cancer Society) there are many challenges to bone marrow transplant. (1) the transplanted cells may not be able to graft into the host's bone marrow. (2) the graft may attack the host in a “graft-versus-host reaction.” (3) The risk of infection is increased before the donated cells become mature enough.

We, therefore, will conduct the experiment in three parts: Experiment 1A will show here that the intravenous administration of protein nanoparticles (whether the nanoparticles come from FPS which is a suspension of FAS with the proper excipients, or just the FAS in saline) to a healthy donor can increase the concentration of cells which can later be used for transplantation to a compatible recipient to increase the probability of success because of the increased mass of donor cells, or a more needed population of cells (needed by the recipient after total ablation) which are produced in the donor due to the positive influence of the nanoparticles.

Experiment 1B will show the effect of the intravenous administration of protein nanoparticles into the recipient after receiving a standard dose of pluripotent cells extracted from the bone marrow of a healthy donor (who did not receive any nanoparticles.) The increased medical benefits come from the increased number of useful cells which are the progeny of the initially transplanted cells.

Experiment 1C will show the effect of the intravenous administration of protein nanoparticles into (a) the donor before his bone marrow cells (increased in mass or appropriateness for transplant) are harvested; and also (b) into the recipient after he received the “improved” version of donor cells.

We expect that any of the above approaches (1A, 1B, or !C) will result in the decrease in the rate of failure of grafting, or the occurrence of infection. We also expect that there will be positive effects of nanoparticle administration in the reduction of graft-versus-host reactions.

Due to the technical aspects of the design of the experiment, we cannot measure directly the number of donor cells that survive in the host. We can only measure the concentration of the cells that are the progeny of the initially infused cells. It can be easily understood that if one were to choose between (a) the number of transplanted cells that can graft successfully right away after the transplant event, vs (b) the number of progeny cells that eventually can settle in the bone marrow and produce useful differentiated cells useful to the host, situation (b) would be preferable; although it is possible that the fact that we have situation (b) is due to the existence of situation (a). Due to the timing of the administration of FAS with respect to the timing of the transplant event (administration of donor cells into the recipient), we can only assess the difference between the FAS-group and the control (saline)-group with respect to situation (b), with the understanding that situation (a) is probably the reason why situation (b) has occurred.

### Material and Methods:

FPS was manufactured according to the published work of Yen (“Mass Production of Ready-to-use Suspensions of Fibrinogen-coated Albumin Spheres for the treatment of Thrombocytopenia Patients” USPTO publication number: 20160354481, published Dec. 8, 2016).

The experimental design is as follows:
Experiment 1A: Mice with Green Fluorescent Protein (GFP) genetically integrated into their genome (e.g. Jackson lab, stock number 006567) were used as donors. They were administered one dose of FAS (16 mg per Kg, i.v.) at various times before they were sacrificed to provide bone marrow cells for transplant into non-GFP mice after the recipients received total bone marrow ablation. The control group was administered 2 mL of saline per kg. The time of FAS or saline administration was day −3, −7, −14 before the harvest of GFP-stem cells from their bone marrow (on Day 0). The percentage of success of the transplant into the recipients was monitored; the concentration of GFP-blood cells after successful transplant (CBC with differential) were measured; all organs were harvested for measurement of GFP-cells incorporation into the organs; bone marrows were studied in the recipients for the percentage of cells that were GFP positive.

For Experiment 1B, the recipients received the FAS. Donor mice and recipient mice were the same as described in Experiment 1A. For recipients, we subject healthy mice (no wounds or dysfunctional organs) to total bone marrow ablation, using the protocols in the literature, as done in Experiment 1A. We then divide the mice into 6 groups (n=6) which would receive transfusion of GFP-bone marrow cells and FAS as shown in the following Table 1, all administered intravenously. The dose of FAS (16 mg per kg) is given only once at the time shown in Table 1. Similar to Experiment 1A, the donor bone marrow cells were harvested from mice purchased from Jackson lab, stock number 006567, strain name is C57BL/6-Tg (CAG-EGFP) 131) sb/LySopJ. All the cells express the Green Fluorescent Protein (GFP). The Jackson Lab website provides detailed information on the percent of cells in various subgroups (e.g. B cells, eosinophils, neutrophils etc.) that are “green”—all of which typically have over 95% of the cells being green. Therefore, the progeny of the donor cells can be traced in all the major organs including the blood in the compatible non-GFP recipient mice.

TABLE 1
Administration of FAS and donor GFP-bone marrow
cells in totally bone-marrow-ablated mice

| | | EXPECTATIONS IN | |
|---|---|---|---|
| | | BONE MARROW OF | EXPECTATIONS IN |
| | TIMING OF FAS | RECIPIENT (6 WEEK | MAJOR ORGANS (12 |
| GROUP | ADMINISTRATION | POST INFUSION) | WEEK POST INFUSION) |
| A | No FAS. Saline (2 mL | This group provides the | This group provides data |
| | per kg) and donor cells | baseline: noticeable | on % green cells in organs |
| | infused into the host 4 hr | fluorescent cells inside | (including blood cells and |
| | after saline | marrow | skin) that form the baseline |
| B | FAS pre-mixed with | About the same as Group | Possibly more fluorescent |
| | bone marrow cells in bag | A | cells in practically all |
| | in vitro, before infusion | | organs than Group A |
| | to host | | |
| C | FAS infused 4 hrs before | Slightly more fluorescent | Obviously more |
| | bone marrow cells | cells than Group A | fluorescent cells in |
| | | | practically all organs than |
| | | | Group A |
| D | FAS infused 4 hrs after | More fluorescent cells | Possibly more fluorescent |
| | bone marrow cells | than Group A | cells in certain mature cell |
| | infusion | | lineages, e.g. white blood |
| | | | cell subgroups |
| E | FAS infused 2 weeks | More than double the | Definitely more fluorescent |
| | after infusion of bone | number of fluorescent | cells in many cell lineages |
| | marrow cells (Half-way | cells than Group A | |
| | to complete grafting of | | |
| | donor cells) | | |
| F | FAS infused 4 weeks | Slightly fewer fluorescent | About the same as Group E |
| | after infusion of bone | cells than Group E | |
| | marrow cells (After | | |
| | almost-complete grafting | | |
| | of donor cells) | | |

The dose of bone marrow cells that will result in successful grafting has been published. We use the guidelines disclosed in various published papers. (1) “The typical yield of bone marrow cells from both femurs and tibia per 8-12 week old C57BL/6 mouse is about 60 to 80 million cells with a viability of 94 to 98%” (“Isolation, Purification and Labeling of Mouse Bone Marrow Neutrophils for Functional Studies and Adoptive Transfer Experiments” J. Vis Exp. 2013; (77):50586, author Swamydas et al.) (2) the number of bone marrow cells needed for successful grafting is about 500 million marrow cells per kg of the recipient (“Technique for Human Marrow Grafting” Blood vol 36, Issue 4, October 970, pages 507-515.) Since the average weight of one mouse is 30 gram, the number of bone marrow cells needed is 500×0.03, or 15 million cells per mouse. Assuming the number of cells needed for successful grafting is similar between mouse and humans, the bone marrow cells harvested from one mouse is sufficient for transfusion into 4 mice. For each group A to F, the number of animals is 6; therefore, we have 36 recipients and we sacrificed 9 GFP-containing mice from Jackson Lab to obtain their GFP-containing bone marrow cells.

Another published method is to use CD34+ bone marrow cells. Among isolated bone marrow cells, the percentage of CD34+ cells are only 1 to 2%. That means we can expect only 1-2% of an average of 70 million cells, i.e. about 1 million cells from the mouse bone marrow to be CD34+ cells. However, in human patients, clinical experience shows that the minimum number of CD34+ cells needed for engraftment is generally agreed to be at least 2.5 million cells (per patient), although an additional infusion of 5 million CD34+ cells is better (“What is the Optimal Number of CD34+ peripheral blood stem cells for an autologous transplant?” Jillela et al, at Stem Cells Dev. 2004 December, vol 13, No. 6). Due to the uncertainty of the different needs between the two species, and the technical challenge of first harvesting CE34+ cells from bone marrow, and then transfusion of these cells to the recipients, we opted to use “total number of (all the isolated) bone marrow cells” as described in the above paragraph.

After the bone marrow transplant event, mice were sacrificed at 6 weeks (3 mice from each group) for the bone marrow to be studied, and 12 week (3 mice per group) for all the major tissues (hematological and non-hematological) and essential organs of the body to be studied. Specifically, we want to measure the percentage of stem cells that are fluorescent in the bone marrow for each group and the distribution of mature fluorescent cells among all the blood cells, and in the various organs. The organs studied include and not limited to the skin, the epithelial cells from the colon and the small intestine, bronchial epithelial cells, alveolar macrophages, adipocytes. A list of organs and tissues with their turn-over rate and their mass in turn-over rate has been published in “The Distribution of Cellular Turnover in the Human Body” by Sender and Milo, in Nature Medicine vol 27, January 2021, page 45-48. We study the presence of GFP cells in the relatively high turn-over tissues because they are most likely to accept the progeny of the grafted bone marrow cells which have the GFP inside. This does not rule out the possibility that “stable tissues” such as neurons can benefit from progenies of the grafted bone marrow cells, even though their turn-over is slow.

For Experiment 1C: the optimal condition of Experiment 1A and Experiment 1B were used. The results were studied similar to those in Experiment 1A and 1B.

### Results:

All three experiments (1A, 1B, 1C) showed positive results in that FAS-treated mice received more medical benefit than the control saline-treated mice. For specific data particularly that of Experiment 1B, the expectations of each group are listed in Table 1 for convenience of comparison between groups. In addition to the tissues mentioned above, the major organs studied by serial sectioning (and then staining with anti-GFP antibody linked to an enzyme) include: lung, heart, liver, pancreas, muscles, brain, skin, blood, intestine. Other organs were also harvested and frozen for more detailed study later. The histological studies of the bone marrow for fluorescent cells and on the major organs harvested in week 12 were consistent with the expectations listed in Table 1.

### Comments:

The administration of FAS manufactured with the published mass production method provided medical benefits in all three Experiments. Overall, FAS has the effect of (a) enriching the concentration of bone marrow cells in the donor and the number of engrafted cells in the bone marrow of the recipients; (b) producing mature progeny cells in the recipient which can populate the existing tissues and organs; however, the benefit appears to be mostly in the hematopoietic cell lineage. This suggests that cancer patients with blood cancers who need bone marrow transplant who have difficulties in obtaining perfectly matched donors may benefit from the administration of FAS in combination with bone marrow transplant, because without the administration of FAS the success rate of such transfusions would be very low. The best timing in human patients with respect to the administration of FAS versus bone marrow donor cells will need to be evaluated in human clinical trials. More detailed studies of the frozen sample in all of the organs from the bone marrow transplant recipient may show that there is a difference between Group A and all the other Groups in Experiment 1B: the data will guide the administration of FAS and the donor cells with greater benefit to specific patient groups who are not cancer patients but have other issues that can benefit from increased concentration of specific lineages of stem cells.

The donor cells in this experiment were harvested from the bone marrow. However, it is now known that various other tissues have stem cells. We will repeat this experiment with stem cells harvested from the peripheral blood, umbilical cord, muscles, or from the adipose tissue. The methods to isolated stem cells from peripheral blood or umbilical cord can be found in the published literature. For example, the reference for isolating stem cells from the muscle and adipose tissues, respectively, are: “Hematopoietic Potential of Stem Cells Isolated from Murine Skeletal Muscle” by Jackson et al, at Proc. Natl Acad Sci USA. 1999 Dec. 7; 96(25):14482-14486, and “Adipose-derived Stem Cells: Isolation, Expansion and Differentiation” by Bunnell et al, at Methods, 2008 June: 45(2):115-120.

We expect the results from stem cells/pluripotent cells harvested from other tissues to be equally effective as those harvested from the bone marrow and that their efficacy can be enhanced by the administration of FAS as illustrated from Experiment 1A, 1B, 1C above.

### Experiment Two:

Effect of protein nanoparticle administration on the development of grafted bone marrow cells in a “partial ablation” recipient/host

### Introduction:

In some of the patients who need bone marrow transplant, it may be advantageous to do only a partial ablation or “mini-ablation” before the infusion of the donor cells. The main advantage is that the patient will have some of his bone marrow cells intact that can produce white cells (although in reduced numbers) to fight against infection during the time the donor cells need to establish themselves. The obvious disadvantage is the possibility of not having killed all the cancer cells which would more likely occur with a “full ablation” of the bone marrow. However, recently discoveries have found that the donor cells can retain their immunity and can kill any residual cancer cells from the host, which are left over from the partial ablation procedures. The purpose of this experiment is to evaluate if the transfusion of FAS is equally effective in a partial ablation animal model as previously demonstrated (Experiment 1) in a total-ablation model. We will also investigate if the application of FAS in conjunction with donor cells would promote a greater (or less) capacity of the donor cells to kill residual cancer cells in the host.

### Materials and Method:

The FAS is taken from the same lot as Experiment One and the protocol for Group E is used here except that the dose of chemotherapy/radiation is designed to achieve only partial ablation of the host's bone marrow. The mice were divided into two groups: E1 being the saline-control; E2 being the FAS group (16 mg per kg). None of the mice had cancer.

### Results:

The percentage of fluorescent cells in the bone marrow and in the mature organs were reduced compared to the data in Experiment One. However, when the data from E2 is compared to E1, it is obvious that there are more fluorescent cells in E2 than in E1, in the bone marrow as well as in the mature cells.

### Comments:

The data will show that the administration of FAS is effective in increasing the success of engrafting of donor cells from a donor (whether GFP-cells or non-GFP-cells) leading to a higher percentage of the donor cells being able to survive in the bone marrow of the recipient and to become mature cells later, compared to the saline-control group.

Although this Experiment Two did not use mice with cancer, we expect the same medical benefit to be conferred from the administration of FAS (whether to the donor, or to the recipient, or to both donor and recipient) in hosts with cancers. The administration of FAS in these transplant cases also will not negative impact the ability of chemotherapy or radiation to kill cancer cells, nor would FAS promote the growth of cancer cells.

We also expect all the donor pluripotent cells (whether from peripheral blood, umbilical cord, muscles, adipose tissues), after treatment with FAS, can be effective than the standard methods without FAS, if not more effective in the ability to kill a large portion (if not all) of the residual cancer cells in the host/recipient who received partial myeloablation.

We are aware that in many cancer cases, the oncologist will choose to use a non-ablation method on the host/recipient, and the method will utilize the graft-vs-host immune response directed against minor histocompatibility antigens. See: “Non-myeloablative Transplantation” by Maloney et al, published in Hematology Am Soc Hematol Educ Program: 2002:392-421. We expect the medical benefits of FAS will be extended to such methods of cancer treatment.

### Experiment Three:

Effect of protein nanoparticle administration on the development of transplanted bone marrow cells in a “total ablation” host who also suffer from a wound or defects at a location away from the bone marrow site.

### Introduction:

Yen has demonstrated in an otherwise-healthy (non-ablated) host which has a irradiation-induced skin wound, that the administration of FAS can lead to accelerated healing of the skin wound when the FAS was administered (a) prophylactically, before the irradiation event; (b) as a mitigation agent, when the FAS was administered immediately after the irradiation event; and also (c) as a therapeutic agent when FAS was administered at a later time, e.g. after the appearance of the skin lesion, typically a month or more after the irradiation event. Given the possibility that a wound can have a stimulatory effect on the stem cell production and that a “channel effect” may occur, i.e. more stem cells may develop into cells that can later differentiate into mature cells helpful to heal that wound (instead of wasting energy in making cells of “general use” and not focused on the existing wound), the experiment here is aimed at discovering if the existence of a wound can affect the effectiveness of FAS in terms of channeling the stem cells toward a most-productive pathway of development (i.e. toward what the host needs at the moment.)

### Materials and Methods:

Protocol E and materials used in Experiment One are used here. The wound is produced by irradiation to the lung at a dose sufficient to result in lung fibrosis. Yen has filed a provisional patent application related to this experimental procedure, titled “Protein Nanospheres to Treat Pulmonary Injury and Dysfunction”. The document was sent by Priority Mail on Aug. 17, 2023, tracking number 9505 5265 5446 3229 8917 38.)

In this experiment, the irradiation event to the lung (causing the harm) is done one week after infusion of the FAS (i.e. in week 3 after the infusion of the GFP-donor cells) so that there is time for the stimulatory effect (if any) of FAS to the implanted bone marrow cells to have occurred. There are two groups of animals: Group G is the saline-control (2 mL per kg); Group H is the FPS-treated group (2 mL per kg, same as 16 mg spheres per kg.)

### Results:

The data is pending and we expect them to show that there are more fluorescent cells in the lung biopsies of Group H than in Group G, and the degree of pulmonary fibrosis is less severe in Group H than Group G.

### Comment:

The data reveal that the presence of a wound would stimulate or channel more mature cells to be generated which will arrive at the wound site and the result is a more complete healing of the wound in the group treated with a protein nanoparticle such as the FPS. This is noticeable because the wound in this model is not one discreet wound at one location, like that of a surgical wound or an irradiation-induced skin injury, but a “diffused injury” all over the lung tissues. Irradiation-induced injuries typically would cause damage not only to the blood vessels under the irradiation field, but to a multiple number of tissues. The recovery of all these tissues is consistent with the fact that stem cells have arrived at each of the different tissues, resulting in differentiation of the stem cells into a variety of the healthy tissues at each location to restore the function of the entire organ, which is in this case the lung.

### Experiment Four:

Effect of protein nanoparticle administration on the development of grafted bone marrow cells in a “total ablation” host who also suffer from more than one wound or defects at a locate away from the bone marrow site.

### Introduction:

One constant worry concerning new technologies to stimulate the bone marrow cells is the potential that the stimulation by the new agent on the bone marrow may “deplete” the reservoir of stem cells in the bone marrow leading to a condition like aplastic anemia where the bone marrow cannot function normally in later life to sustain the need of the body to replenish old cells with new cells. This experiment is aimed at assessing if the supply of stem cells is still adequate when the body is challenged with more than one injury, such as caused by injection of a muscle-destroying agent (glycerol) that will also cause kidney dysfunction.

### Methods and Material:

The protocols of Experiment Three are used here, except that the wound is not irradiation-induced pulmonary injury, but glycerol-induced rhabdomyolysis leading to kidney failure, as described in a previous Provisional patent application (“Protein Nanospheres to Treat Renal Dysfunction” mailed to the USPTO on Jul. 28, 2023 with a USPS tracking number of 9505 5265 5446 3209 8704 49). Because there are multiple injuries, the model fits into the evaluation on the effectiveness of protein nanospheres or nanoparticles in the treatment of polytrauma. The muscle injury is performed one week after the infusion of FAS intravenously into the mice; i.e. FAS was infused into the bone marrow-depleted mice 2 weeks after the ablation; then muscle injury was performed in week 3.

### Results:

Group J is the saline-control group (2 mL per kg); Group K is the FPS-group (16 mg spheres/kg). The data is pending and we expect them to show that healing of both the muscle site and the kidney dysfunction in Group K is better than in Group J. There are definitely “green cells” in the kidney and the muscles of both groups, but more so in the FPS-treated group than the saline-group. However, histological examination of the bone marrow for green bone marrow cells shows that the percentage of green cells is about the same between Group J and Group K. Therefore, there is no evidence that the infusion of FAS during a time when the bone marrow had not been fully replenished would deplete the source of stem cells for later mobilization in the context of a need to heal more than one injury at the same time.

### Comment:

This model uses bone marrow cells that had incorporated into their DNA the fluorescent gene for tracking. Therefore, the donor cells are implanted from an external source. The data support the hypothesis that donor bone marrow cells can differentiate later into mature (green) cells to result in the healing of more than one injured site. The data do not exclude participation of the host's own residual bone marrow cells which could also differentiate into similar mature cells towards the healing of the injured sites (mainly because the host's own bone marrow cells cannot be traced, being “non-green.”)

The data here confirm the previous finding in Yen's Provisional Patent Application “Protein Nanospheres to Treat Renal Dysfunction” that in an “otherwise-healthy”, non-transplant patient (i.e. patient who has multiple injuries but did not receive any bone marrow transplant) the administration of FAS will stimulate stem cell production at the bone marrow site (or any other site of origin for cells that are pluripotent) for differentiation into mature cells capable of healing more than one injury at the same time—but the data here add weight to the proposition that the bone marrow can do so without any evidence of deletion of the stem cells. The other possibility is that in a host with multiple injuries and who has not received bone marrow ablation, donor bone marrow cells or stem cells can still be infused (without ablation of the host's bone marrow) which will further accelerate the process of healing multiple wounds.

### Experiment Five:

Effect of protein nanoparticle administration on the development of transplanted bone marrow cells in a “total ablation” host who also received a blood cancer cell line to evaluate if the effect of FAS on the bone marrow may include stimulation of the growth of cancer cells.

### Introduction:

The aim of chemotherapy is to kill all the cancer cells (if possible) without (if possible) harming the healthy cells. In a situation where the cancer cells appear to be resistant, bone marrow ablation in these cancer patients is performed knowing that the vital bone marrow cells would also be eliminated alongside with the cancer cells. The patient is then infused with donor bone marrow cells or other compatible stem cells to replenish the depleted bone marrow and to restore the ability to form new (normal) cells. It is therefore important to ascertain that the effect of FAS on the bone marrow does not include the possibility of stimulating cancer growth.

Regarding leukemia stem cells there is an excellent article published in Biomedicines. 2018 March; 6(1): 22. The title is “Targeting Leukemia Stem Cells in the Bone Marrow Niche” authored by Sarah K. Tasian, et al. The abstract stated: “The bone marrow (BM) niche encompasses multiple cells of mesenchymal and hematopoietic origin and represents a unique microenvironment that is poised to maintain hematopoietic stem cells. In addition to its role as a primary lymphoid organ through the support of lymphoid development, the BM hosts various mature lymphoid cell types, including naïve T cells, memory T cells and plasma cells, as well as mature myeloid elements such as monocyte/macrophages and neutrophils, all of which are crucially important to control leukemia initiation and progression. The BM niche provides an attractive milieu for tumor cell colonization given its ability to provide signals which accelerate tumor cell proliferation and facilitate tumor cell survival. Cancer stem cells (CSCs) share phenotypic and functional features with normal counterparts from the tissue of origin of the tumor and can self-renew, differentiate and initiate tumor formation. CSCs possess a distinct immunological profile compared with the bulk population of tumor cells and have evolved complex strategies to suppress immune responses through multiple mechanisms, including the release of soluble factors and the over-expression of molecules implicated in cancer immune evasion. This chapter discusses the latest advancements in understanding of the immunological BM niche and highlights current and future immunotherapeutic strategies to target leukemia CSCs and overcome therapeutic resistance in the clinic.” These published statements reinforce the complexity within the bone marrow and the challenges facing clinicians who want to kill the cancer cells without doing much harm to the normal cells.

Given the complexity of the leukemia stem cell biology, we intend to use a widely used mouse cell line as a pilot study to evaluate the effect of protein nanoparticles on the (lack of stimulatory effect on) development and suppression of cells that target the bone marrow. We will use the mouse cell line called C1498 (ATCC® TIB-49TM).

According to the article titled “C1498: A murine model for acute myelogenous leukemia (AML)” authored by Dylan Daniel, published in Biopharma/labcorp.com in April 2017 (https://biopharma.labcorp.com/industry-solutions/by-therapeutic-area/oncology/preclinical/tumor-spotlights/c1498-a-murine-model-for-acute-myelogenous-leukemia-aml.html): “C1498 is a murine myeloid leukemia cell line isolated from a C57BL/6J mouse. It most closely resembles acute myeloid leukemia (AML) and is highly lethal when implanted into syngeneic mice.” We will implant the optimal number of C1498 cells into its syngeneic mice which had been bone-marrow-depleted and re-implanted with GFP-gene bone marrow cells, followed by either administration of saline or FAS in an environment of C1498 invasion. The goal is not the elimination of the cancer cells, but to assess if the FAS-group would create an environment which promotes faster or more toxic cancer growth than in the saline-group.

In terms of treatment for leukemia stem cells, numerous treatments have been proposed and tried for this complex web of related cancers. For example, Tasian et al stated: “Strategies for targeting leukemia stem cells (LSCs) fall into two broad categories: therapies that eradicate LSCs (termed “LSC-specific”) and therapies that eradicate both the bulk of AML and the LSC compartment (termed “LSC-active”). The first defined LSC-specific immunophenotypic property was expression of CD123 within the CD34+CD38-compartment. Some of the differentially expressed molecules are being targeted in pre-clinical models of hematological malignancies and in clinical trials, mostly using antibody-based and cell-based therapeutic approaches. CD123 has been targeted with neutralizing monoclonal antibodies (e.g., 7G3) in NOD/SCID mice [115] and in patients with relapsed/refractory AML. In one study, 7G3 treatment reduced the engraftment potential of AML-derived LSCs and improved mouse survival. 7G3 also inhibited IL-3-mediated intracellular signaling of isolated AML CD34+CD38− cells in vitro and reduced their survival.

In terms of treatment of C1498, the article by Daniel stated: “There are multiple ongoing clinical trials for immunotherapies in AML (acute myeloid leukemia), including more than 15 trials with anti-PD-1 or anti-PD-L1 checkpoint inhibitors. C1498 has been reported to express low levels of PD-L1 in cell culture, but significantly upregulate PD-L1 expression in vivo. The expression of PD-L1 in vivo renders the model sensitive to tumor growth delay by PD-1 blockade. This is accompanied by a significant increase of CD4+ and CD8+ T cell infiltration in AML diseased liver. These published data suggest that C1498-luc-mCherry would be an ideal model to test immunotherapy combinations with checkpoint inhibitors.”

### Material and Methods:

Protocol E of Experiment One is used here. Group L is the saline control and Group M is the FPS treated group. Both groups received a minimum dose of cancer cells derived from the leukemia cell line C1498, infused 3 days before the administration of FAS.

### Results:

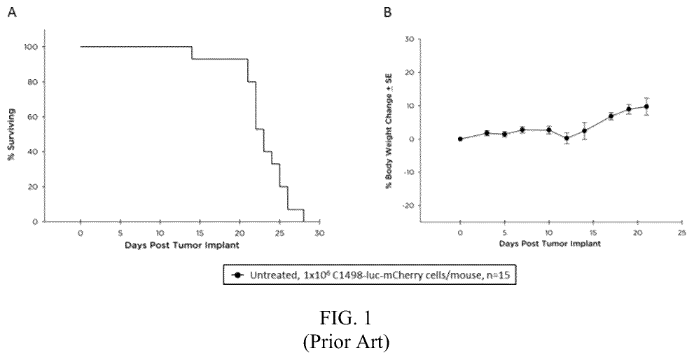

For reference, FIG. 1 is a reproduction of FIG. 2 of the article by Daneil showed: FIG. 2: Survival kinetics (A) and body weight change (B) of C57BL/6 mice with disseminated C1498-luc-mCherry AML.

At this stage we are still trying to purchase the C1498 cells and the medication most optimal for the suppression of these cancer cells in vivo. But we expect the % survival of the saline-group to closely resemble that of the FAS-group. The data will show that there is no evidence of stimulation for the growth of the cancer cells in vivo. We also expect that when the optimal chemotherapy or radiation or immune-treatment is rendered to mice who had been implanted with the cancer cells, the effectiveness of the treatment will be essentially similar between the saline-group and the FAS-group.

### Comments:

The data will show that the administration of FAS into a mouse with implanted bone marrow donor cells will not suffer from the administration of FAS, in case there are still cancer cells left in the body, when compared to the control group.

Part Two: Using FAS as a Separation Tool In Vitro and In Vivo to Isolate “Special” Lineages of Pluripotent Cells from the Other “Overall” Cell Population Extracted from the Bone Marrow or Elsewhere, to Evaluate (1) the Engraftment Efficiency of these Isolated “Special” Population of Cells; (2) Whether the Isolated “Special” Population Will Produce Progeny Cells Useful for Specific Purposes Compared to Progeny from Unselected Pluripotent Cell Populations

### Experiment Six:

Selection of natural stem/progenitor cell populations in vivo, by protein nanoparticles for more successful grafting or specialized differentiation.

### Introduction:

The history of the development of FPS as a stem cell mobilizer and now as a stem cell “selective population selector” follows a non-obvious sequence of events, which took many steps/experiments to connect together.
(1) FPS initially was developed as an “artificial platelet.” (“Fibrinogen-coated microspheres” U.S. Pat. No. 6,264,988, issued Jul. 24, 2001.) That invention was based on the observation that the key biochemical step of the activation of platelets is the binding of fibrinogen molecules onto the surface of platelets which were previously non-activated, endogenous, and free flowing “particles” inside blood vessels. Thus, a nanometer-sized biocompatible sphere coated with fibrinogen may mimic activated platelets and it would be passively trapped by the mass of platelets as they are activated at wound sites inside the blood vessel; specifically along the wall of the vessels without forming obstructive clots in the middle of the blood vessel, or impeding the blood flow. These “fibrinogen-coated albumin spheres” would form co-aggregates with activated platelets due to the continued action of enzymes such as thrombin, which are located at the wound site on the endothelium-leading to a large therapeutic bulk capable of plugging the wound in a timely manner, especially in thrombocytopeniatients, wherever the wounds are located along the blood vessels. There would be no free-floating aggregates moving downstream from the wound site, because all the enzymes needed for the activation and aggregations of platelets are tightly bound to the wound site and not free-floating in the blood stream.
(2) There was great interest in finding out whether control of bleeding would be helpful to the survival of patients subjected to lethal doses of irradiation, such as would occur in an atomic war or in a nuclear industrial accident. To everybody's surprise and delight, administration of FAS in a lethal-irradiation mouse model revealed that survival can be increased from 5% in some cases to 60%: the mechanism is without a doubt the control of internal bleeding which was caused by the extensive damage of irradiation to all the blood vessels (Sung, Yen, et al, “Fibrinogen-Coated Albumin Nanospheres Prevent Thrombocytopenia-Related Bleeding” Radiat Res. 2020 Aug. 1; 194(2): 162-172.) Also, “Submicron particles for the treatment of radiation damage in patients” U.S. Pat. No. 9,226,898, issued Jan. 5, 2016. However, in retrospect, the question can be asked: “Timely control of internal bleeding will only reduce the volume of blood loss so that the animals do not die from internal bleeding, but all the cells in the body have been irradiated with a high dose of irradiation—how did they recover from the lethal injuries induced by the irradiation?”
(3) Experiments to evaluate the improvement of survival by FPS after lethal doses of whole-body irradiation would involve the irradiation of hundreds of mice. In addition, the technique of irradiation is challenging—a slightly higher dose of irradiation can lead to 100% mortality, and a slightly lower dose of irradiation would lead to 100% survival—these conditions make it hard to evaluate the positive effects of FPS. Therefore, there is incentive to design an “easier” system for evaluation. It was thought that a local dose of irradiation to the skin (performed when the animals are under anesthesia) would be easier to conduct than to apply whole-body irradiation to a large number of (roaming) animals. The data showed that FPS is effective in accelerating the healing of skin injuries when administered (a) before the irradiation event, (b) immediately after the irradiation event, (c) even after the appearance of the skin lesion which occurs many weeks after the irradiation event. The result was a surprise because the major effect of a high dose of irradiation is the complete destruction of blood vessels under the area of irradiation. The question was raised: “If there was no blood supply to the area after irradiation, then how could the spheres arrive at that site to promote healing?” The mechanism of healing, therefore, cannot be the formation of co-aggregates of the fibrinogen-coated albumin spheres with activated platelets. There must be another mechanism, which occurs at a site away from the site of irradiation. But what mechanism and where? Moreover, it was observed that skin injury involves a large number of tissues which would all be injured, including not just the skin, but the subcutaneous tissues such as fat, muscles, blood vessels, nerves. These different tissues all appear to have coordinated recovery from one dose of FPS—this leads to the hypothesis that “stem cells must have been involved” because stem cells (which originate from the bone marrow and have gone into the blood stream) have the pluripotent capacity to differentiate into different tissues depending on where they land. Histological studies of the biopsies obtained from the healing skin lesion confirm the hypothesis: stem cells (CD34+) were in abundance in the FPS-treated group, far more than in the saline-treated group. In cases where neo-vascularization was studied, CD31+ cells were in greater abundance in the FPS group compared to the control saline group: CD31+ cells being endothelial cells lining the blood vessels. Standard staining for the tissues also showed that blood vessels have recovered completely in the FPS group and only very poorly in the control (saline) group.
(4) Since the most abundant site of stem cells is the bone marrow, an effort was made to study how the intravenously administered FPS interacts with cells in the bone marrow. The fibrinogen-coated albumin spheres (FAS) were first labeled with fluorescent molecules (Fluorescein Isothiocyanate, FITC). Such labeling steps do not affect the function of the FAS but they allow visualization of where the spheres are located. One dose of FITC-FAS administered intravenously was shown to have traversed from the intravenous compartment into the bone marrow compartment of the bone marrow. The data showed that bone marrow cells isolated from the bone marrow after such an intravenous infusion of FITC-FPS still have FITC-FAS attached, despite the vigor of the separation steps involved to isolate the bone marrow cells from the bone marrow. In addition, non-FITC-FAS, i.e. the original FAS, was shown to increase the concentration of CD34+ cells later inside the bone marrow, more so in FAS-group when compared to the saline-group.

The combination of the above observations strongly suggest but does not prove that the stem cell mobilization process involves the following steps: (a) movement of FAS from the intravenous compartment into the bone marrow compartment; (b) attachment of FAS to some or all the relevant stem cells or mesenchymal cells, which probably send a signal to these pluripotent cells to reproduce; (c) exit of the FAS-attached cells (or cells which might have detached the FAS by now) from the marrow; we do not yet know if the attachment of FAS to cells inside the bone marrow is related to any stimulation or activation of this population of bone marrow cells that have interacted with the FAS; (d) further maturation of this group of cells in the peripheral blood (it could be a single lineage of cells or a multiple number of lineages of cells); (e) arrival of the stem cells at the target site (e.g. wound sites at the skin or internal organs, e.g. the gastrointestinal tract or the brain, after a high dose of irradiation): this shows up as CD34+ cells at the target site; (f) the abundance of CD34+ stem cells at the target site is highly correlated with the observation of accelerated healing macroscopically/histologically: in order to have the functional cells at the target site to do the healing, stem cells must differentiate into the various mature tissues. Mature cells typically shed the CD34+ cell markers. The abundance of CD34+ cells at the target site at a late stage means there was an over-abundance of CD34+ cells that have arrived. Despite the need to differentiate into non-CD34+ (mature) cells, there are still plenty of CD34+ (primitive) cells left at the site. There is a real possibility that in some models of wound healing, all of the “originally CD34+” cells could have all differentiated into mature, “non-CD34+ cells”—thus leaving no detectable CD34+ cells at the site in a later time frame (when the injury has healed.) This is verified in the mini-pig model (NIH grant 2R44Al142964-02 “A multivalent easy-to-use product to mitigate and treat radiation exposure”). Staining for both CD34+ cells and CD31+ cells (which are mainly located in the endothelium of blood vessels) in the mini-pig skin biopsies showed that at the time of biopsy (collected at many weeks after the irradiation event) there was an abundance of CD31+ cells consistent with the histological observation that neo-vascularization is almost completed in the FAS-group compared to the control group. However, for the CD34+ cells, there was no statistically significant increase of CE34+ cells in the FAS-group in the healed tissues compared to the saline-group—all the cells that initially arrived at the wound which might have been CD34+ must have all shed the CD34 marker as they mature into the functional cells of the various tissues in the healed would site by the time of biopsy.

We stated at the beginning of the previous paragraph that the independent evets (“a” to “f”) strongly suggest but “do not prove.” This is because of the preconceived idea that event “a” could have or should have led to event “f” from other known biological events. Any critics could have argued for argument's sake that the attachment of FITC-FAS (event “b”) could lead to the death of the attached cell from the bone marrow instead of its stimulation. To show that the attachment of FITC-FAS to a bone marrow cell would lead to propagation (or division) of the attached cells would require a “lineage study”. Such a lineage study needs to show that the same bone marrow cell that had been attached with FITC-FAS actually survived and became (differentiated into) the mature skin cells which were found later at the skin lesion site. So far, no lineage studies have been done and it is not obvious at this time whether the attachment of FITC-FAS to a bone marrow cell is harmful or beneficial to that bone marrow cell. Without such lineage studies, putting the events of (a) together with (b) and (c) all the way to event (f) is speculation and not proof of their biological relatedness. If event (b) is harmful to the bone marrow cell, that could be a terminal event, leading to none of the events such as (c) or (d) m far less all the way to event (f). Therefore, the present experiment is designed to evaluate the possibility of using FAS to isolate bone marrow cells (in vivo and in vitro) which will allow us to follow the fate of these specific cells, with or without signals from a wound to inform these stem cells the need for accelerated healing due to the presence of a wound.

In addition, the previous experimental data (events “a” to “f”) tell only one one-sided story. The fact that FAS introduced intravenously can attach to certain cells in the bone marrow in vivo does not mean (a) that all bone marrow cells are capable of attaching to FAS in vivo; or (b) that the cells that can bind to FAS in vivo are similar to cells that can bind FAS in vitro; or (c) that any of the bone marrow cells (of which there are many kinds of cells) are capable of attachment to FAS in vitro at all. We, therefore, ask the question in this set of experiments whether any cells in the native populations of stem/progenitor cells can attach to FAS in vitro and be subsequently selected in vitro (and separated from non-attaching cells), and whether such selected population of cells can differentiate into separate lineages of mature cells after successful grafting into a compatible host.

### Purpose:

To evaluate the possibility that the subgroup of bone marrow cells to which FITC-FAS have attached in vivo, can be used as “specialized bone marrow cells” for successful bone marrow transplantation and whether the progeny of these cells can perform specialized functions later in the host

### Materials and Methods:

We intend to use native populations of stem/progenitor cells from any one of the following sources: (a) cells separated from the bone marrow; (b) cells isolated from the endothelium (or endothelial cell lines); (c) cells obtained from flushing various organs, i.e. perivascular cells. In addition, we intend to isolate stem cells from the peripheral blood, umbilical cord, muscles and adipose tissues.

The experimental conditions here are different from those of Experiment One. In Experiment One, condition B, we mixed FAS in vitro with the donor bone marrow cells. Moreover, the mixing was done in the transfusion bag within one hour of transfusion. Here we will administer the FITC-FAS intravenously and allow the binding of FITC-FAS to the “special bone marrow cells” in vivo.

Even though we intend to extend the study to cells in the endothelium and from the perivascular regions of various organs, and from the other tissues as mentioned above, we will start with cells obtained from the bone marrow.

We will start with mice which have been incorporated into their DNA a gene capable of producing “ROSA” fluorescence in every cell in the body. We used the “ROSA” cells so that they can be distinguished from the green fluorescence of the FITC-FAS. The Jackson laboratory sells one such strain of “ROSA mice”, as described in their website:

“B6.129 (Cg)-Gt(ROSA)26Sor/J Strain #007676| Common Name: mT/mG, mTmG mT/mG (also called ROSA) is a cell membrane-targeted, two-color fluorescent Cre-reporter allele. Prior to Cre recombination, cell membranelocalized tdTomato (mT) fluorescence expression is widespread in cells/tissues. Cre recombinase expressing cells (and future cell lineages derived from these cells) have cell membrane-localized EGFP (mG) fluorescence expression replacing the red fluorescence. In addition to this C57BL/6J congenic mT/mG strain (Stock No. 007676), the mT/mG reporter allele is also available on a NOD/ShiLtJ congenic background (Stock No. 037456) and a 129;CD1 mixed genetic background (Stock No. 007576). The mT/mG reporter allele functions similarly to the ROSA reporter allele (nT/nG; Stock Nos. 023035/023537), with mT/mG directed to cell membrane and nT/nG directed to nuclei. Detailed Description Mice homozygous for this mT/mG mutation are viable and fertile. These mice possess loxP sites on either side of a membrane-targeted tdTomato (mT) cassette and express strong red fluorescence in all tissues and cell types examined. Tail or whole body epifluorescence is sufficient to identify mT/mG mice from wildtype mice. When bred to Cre recombinase expressing mice, the resulting offspring have the mT cassette deleted in the cre expressing tissue(s), allowing expression of the membrane-targeted EGFP (mG) cassette located just downstream. The donating investigator reports that the ACTB promoter allows stronger and persistent expression of the fluorescent proteins (especially in adult cells) compared to the endogenous Gt (ROSA) locus alone. This double-fluorescent system allows direct live visualization of both recombined and non-recombined cells at single cell resolution, offering an internal control for phenotypic analysis of Cre-induced mosaic mutants and providing a second marker for lineage tracing applications. In addition, the localization of fluorescent proteins to membrane structures outlines cell morphology and allows resolution of fine cellular processes. These mT/mG mice are useful as a Cre reporter strain; expressing red fluorescence prior to, and green fluorescence following, Cremediated recombination in widespread cell and tissue types. In an attempt to offer alleles on well-characterized or multiple genetic backgrounds, alleles are frequently moved to a genetic background different from that on which an allele was first characterized. It should be noted that the phenotype could vary from that originally described. We will modify the strain description if necessary as published results.”

The FITC-labeling of FAS is performed according to published methods for soluble proteins. The Certificate of Analysis is as follows in Table 2:

TABLE 2
Various properties of the Preparation containing FITC-FAS

| | | Lot | | |
|---|---|---|---|---|
| Attribute | Acceptance Criteria | Results | | Passed |
| Appearance | Suspension of particles (i.e. | Yes | | X |
| | turbid), brightly yellow to | | | |
| | orange in color | | | |
| Identity | Confirmation of Fibrinogen- | Yes | | X |
| | coated Albumin-based spheres | | | |
| | labeled with FITC | | | |
| Functionality | Formation of sphere- | Yes | | X |
| | augmented fibrin clot | | | |
| pH | pH 6.4 to 7.4 | 7.0 | | X |
| Osmolarity | Not less than 350 mOsm/L | 405 | mOsm/L | X |
| Particle Size | Median diameter <0.5 μm, | 0.1530 um, 0 | | X |
| | and no diameters >5 μm | | | |
| Purity | >90% (% sphere diameters <1 | 99.9% | | X |
| | μm) | | | |
| Potency | >1 activity unit | 5 | a.u. | X |
| Sterility | No Growth | 0 | growth | X |
| Pyrogens | Absence of Pyrogens or | 1.2 | EU/mL | X |

| | | | |
|---|---|---|---|
| | Endotoxin levels <2.9 EU/mL | | |
| Concentra- | 6.5 to 9.5 mg per mL | 8.1 mg | X |
| tion: | | per mL, | |
| mg spheres | | suspended | |
| per mL of | | in 0.9% | |
| suspension | | sodium | |
| | | chloride | |
| | | solution | |

Relating to the FITC content in the FTIC-FAS, the following table (table 3) lists some of the properties in the FITC-FAS spheres:

TABLE 3
FITC and Protein concentration

| | | | Supernatant | Whole |
|---|---|---|---|---|
| CONTENT | UNITS | Sphere Fraction | Fraction | Suspension |
| Protein | mg/mL | 8.1 | 0.3 | 8.4 |
| Concentration | | | | |
| FITC | ug/mL | 20.8 | 0.8 | 21.6 |
| Concentration | | | | |
| FITC/mg | ug/mg | 2.57 | n/a | n/a |
| sphere | | | | |

The administration of FITC-FAS intravenously into ROSA mice is essentially the same as described in Yen patent application “Protein Nanospheres to Treat Harm from Multiple Trauma” first filed as a provisional patent application (63 372 883 on Apr. 13, 2022) followed by a non-provisional patent application. Essentially the mice were administered FITC-FAS at a dose of 32 mg per kg (4× that of effective dose for skin injury recovery) intravenously. The bone marrow cells were collected at 24 hours post-FITC-FAS administration.

Two methods were available to collect the FITC-attached bone marrow cells. (1) The Flow Cytometer cell sorter can isolate “fluorescent” cells (by virtue of the attachment of FITC-FAS to these “special” bone marrow cells). Another possibility is to use magnetic spheres (nanometer in size) that have anti-FITC antibody that can attach to FITC molecules, which are present in the FITC-FAS, which have attached to the competent (or special stem) cells. We used the Flow Cytometer Cell Sorter method.

### Results/Comments:

The following is only part of all the data collected. We collected an average of about 70 million bone marrow cells per ROSA mouse, of which about 0.2% were FITC-FAS positive. Note: the attachment of FAS to the bone marrow cells occurs in vivo, in the live mouse, i.e. the binding occurs in vivo in the bone marrow compartment. We expect the FAS binding site on the stem cell (mainly for binding to fibrinogen molecules or their fragments) to be a different site from the CD34 cell marker. We do not know yet what percentage of these FAS+ cells (meaning they attach to FITC-FAS spheres) are CD34+. This is interesting in that the reported percent of CD34+ cells in the bone marrow is about 1 to 2% (“CD34− an overview” in ScienceDirect Topics). Our data here suggests that some FAS+ cells are CD34+ and some FAS+ cells are CD34negative, while some FAS-cells are CD34+ and some FAS-cells are CD34−.

In the next set of experiments we intend to first isolate the bone marrow cells from the bone marrow of these ROSA mice, and then identify those cells in vitro that can also attach to FITC-FAS spheres, for use in subsequent grafting experiments of these “special” populations.

Theoretically there can be 4 different populations (whether in cells that bind FITC-FAS in vivo, or the binding is done in vitro):

TABLE 4
4 sets of pluripotent cells from their sources:

| | Attach to FITC-FAS | |
|---|---|---|
| | spheres | Do not attach to FITC-FAS |
| CD34 labeling | To be called CD34+/FAS+ | To be called CD34+/FAS− |
| positive | | |
| CD34 labeling | To be called CD34−/FAS+ | To be called CD34−/FAS− |
| negative | | |

In the first set of the experiments, we add the FITC-FAS first (green fluorescence) and then the CD34 antibody (fluorescence at wavelengths different from that of FITC and the ROSA wavelengths). If the fluorescence is positive for both FITC and CD34, then these stem cells are CD34+/FAS+ cells.

For theoretical considerations, there is a slight chance that the binding site on the stem cell for FITC-FAS may share some affinity for CD34+ antibody binding: or causing some mutual stereo-hindrance of binding to each other. If that is the case, the binding of the stem cell to FITC-FAS would have decreased the binding of the CD34 antibody. We can test if this is the case by the reverse approach: by adding the CD34 antibody first, followed by FITC-FAS binding. If the two sets of experiments differ in the % of cell in CD34+/FAS+ cells (or any of the % of cells in the four quadrants in the above table) then we know some receptors on these special cells have commonality between the FAS (mainly fibrinogen receptors) binding site and the CD34 antibody binding sites.

The reason why we need to know the distribution of cells among the 4 possibilities above is (a) it is known that CD34+ stem cells mainly (but not exclusively) lead to hematopoietic cells as they mature in the blood; (b) we do not know yet the fate of the FAS+ cells. The fact that some FAS+ cells are CD34-negative suggests that these CD34-negative cells may be the primitive cells that are responsible for the production of progenies that will populate the non-hematopoietic cells, such as mature cells useful in the healing of deep wounds. One such possibility is the progeny that will replenish the endothelium in blood vessels (CD31+)—they are needed in the neo-vascularization of the previously damaged blood vessels due to irradiation.

After these special stem cells were isolated (whether from in vivo or in vitro attachment of FITC-FAS) they would be implanted into competent mice so that the progeny of these FITC-FAS-isolated ROSA stem cells can be tracked. We expect stem cells isolated in vivo and in vitro would have slightly different fates (“channeled”) so that patients with specific diseases can benefit from the specific lineages of progenies derived from the desired pluripotent cells. For example, patients with hematopoietic disease may benefit more from the special stem cells that preferentially differentiate into hematopoietic cells needed by these patients. On the other hand, patients with non-hematopoietic diseases may benefit more from the “more pure” populations of stem cells isolated by this method because these selected cells would preferentially develop into the non-hemopoietic lines of cells needed by these patients.

Part Three: Various Schemes of Using Pluripotent Cells which were Isolated In Vivo (or In Vitro) Via their Interactions with of FAS, so that Specifically Useful Pluripotent Cells can be Isolated and Later Deployed to Achieve Medical Benefits not Possible with Unselected Populations

We will use mice genetically modified with incorporation of the Red Fluorescent Protein (RFP) to provide the “source-cells” which can be isolated from the bone marrow, endothelium, muscles, adipose, peripheral blood or any competent tissue: to be implanted into various hosts. The purpose is to evaluate (a) efficiency of grafting; (b) specificity of progeny cells. The host may or may not have received some injury or may/may not have genetic dispositions (e.g. cancerous state or other genetic diseases) that will interact with the source cells and their progeny.

| | SOURCE-CELLS ISOLATED FROM BONE MARROW OF HEALTHY | | | |
|---|---|---|---|---|
| | RFP-ANIMALS | | | |
| | | | C. First isolate | D. Administration |
| | | | BM cells from | (iv) of FITC- |
| | | B. Isolate the | untreated | FAS to animals |
| | | entire population | animals, then | to allow BM |
| | | of BM cells from | attachment of | cells to attach to |
| | | animals after | FITC-FAS to | FITC-FAS in vivo, |
| | A. Isolate the | animals are | BM cells in | then isolated this |
| | entire population | administered | vitro, followed | population of BM |
| | of BM cells from | FAS | by implantation | in vitro; follow by |
| | animals not | intravenously. | of this special | implantation of |
| | treated with FAS. | No selection | population of | this population of |
| | (These BM cells | (These BM cells | BM cells. (These | BM cells. (These |
| | are to be called | to be called | BM cells to be | BM cells to be |
| HOST | sBM cells.) | fBM cells.) | called ftBM cells.) | called fvBM cells.) |
| 1.1 Non-ablated | A1 | B1 | C1 | D1 |
| healthy animals | | | | |
| 1.2 Partially- | A2 | B2 | C2 | D2 |
| ablated animals | | | | |
| 1.3 Maximally- | A3 | B3 | C3 | D3 |
| ablated animals | | | | |

Panel 1: Using Bone Marrow (BM) cells obtained from healthy Red Fluorescent Protein RFP-animals as “source-cells” to evaluate fate of transplanted cells in non-injured healthy animals.

Where sBM: means standard BM cells; fBM: FAS-stimulated BM cells; ftBM: FAS-isolated cells in vitro; fvBM: in vivo attachment of FITC-FAS followed by isolation of these fvBM cells.

| | SOURCE-CELLS ISOLATED FROM BONE | | | |
|---|---|---|---|---|
| | MARROW OF HEALTHY RFP-ANIMALS | | | |
| | E. sBM | F. fBM | G. ftBM | H. fvBM |
| HOST | cells | cells | cells | cells |
| 1.4 Non-ablated | E4 | F4 | G4 | H4 |
| injured animals | | | | |
| 1.5 Partially- | E5 | F5 | G5 | H5 |
| ablated and | | | | |
| injured animals | | | | |
| 1.6 Maximally- | E6 | F6 | G6 | H6 |
| ablated and | | | | |
| injured animals | | | | |

Panel 2: Using Bone Marrow (BM) cells obtained from healthy RFP-animals as “source-cells” to evaluate fate of transplanted cells in injured animals. The injury can occur before the BM cell implantation, or after implantation.

(Purpose is to see if certain groups of source-cells are more helpful to the healing of the injured site, or whether the injury in the recipient will change the fate of implanted cells as compared to their being implanted into healthy non-injured hosts.)

| | SOURCE-CELLS ISOLATED FROM BONE | | | |
|---|---|---|---|---|
| | MARROW OF INJURED RFP-ANIMALS | | | |
| | J. wBM | K. fBM | L. ftBM | M. fvBM |
| HOST | cells | cells | cells | cells |
| 1.7 Non-ablated | J7 | K7 | L7 | M7 |
| healthy animals | | | | |
| 1.8 Partially- | J8 | K8 | L8 | M8 |
| ablated healthy | | | | |
| animals | | | | |
| 1.9 Maximally- | J9 | K9 | L9 | M9 |
| ablated healthy | | | | |
| animals | | | | |

Panel 3: Using Bone Marrow (BM) cells obtained from injured RFP-animals as “source-cells” to evaluate fate of transplanted cells in healthy animals. The injury in source animals will occur before the BM cells are harvested.

(Purpose is to see if injuries in the source animal will have already channeled the fate of BM cells into particular pathways potentially favorably to the healing in the host animals which are healthy at the time of transplantation which may have injury in the future.)

| | SOURCE-CELLS ISOLATED FROM BONE | | | |
|---|---|---|---|---|
| | MARROW OF INJURED RFP-ANIMALS | | | |
| | N. wBM | P. fBM | Q. ftBM | R. fvBM |
| HOST | cells | cells | cells | cells |
| 1.10 Non- | N10 | P10 | Q10 | R10 |
| ablated injured | | | | |
| animals | | | | |
| 1.11 Partially- | N11 | P11 | Q11 | R11 |
| ablated and | | | | |
| injured animals | | | | |
| 1.12 | N12 | P12 | Q12 | R12 |
| Maximally- | | | | |
| ablated and | | | | |
| injured animals | | | | |

Panel 4: Using Bone Marrow (BM) cells obtained from injured RFP-animals as “source-cells” to evaluate fate of transplanted cells in injured animals. The injury in source animals will occur before the BM cell harvesting.

(Purpose is to see if injuries in the source animal will have already channeled the fate of BM cells into particular pathways favorably to the healing in the host animals which also have the same or different injury.)
Panel 5: same as Panel 1, except the host will receive FAS-boosting administered intravenously, after source BM cell implantation.
Panel 6: same as Panel 2, except the host will receive FAS-boosting administered intravenously, after source BM cell implantation.
Panel 7: same as Panel 3, except the host will receive FAS-boosting administered intravenously, after source BM cell implantation.
Panel 8: same as Panel 4, except the host will receive FAS-boosting administered intravenously, after source BM cell implantation.

In terms of the results to be evaluated, half of the animals in all of Panels 1-8 will be evaluated for the fate of hematopoietic cells after implantation; the other half will be evaluated for the fate of implanted cells into non-hematopoietic tissues: such as GI-tract cells (for treatment of GastroIntestinal diseases), skin, nervous system, pancreatis (for diabetes treatment.) In addition, the effect of channeling using any of the above methods in all of the Panels in wound healing (single or multiple wounds) will be assessed.

In addition, we contemplate using cells from various productive tissues: Panel 9 to 16 will use source cells isolated from organs or tissues other than the Bone Marrow (e.g. from the muscles). It is known and published that FAS attaches to activated platelets, endothelial cells and cells in the bone marrow. It is not obvious that FAS may attach to cells in other organs or tissues: we intend to show that FITC-FAS do attach to cells from tissues and organs other than the endothelium or the bone marrow.

Panel 9: same as Panel 1, except the “source-cells” will be isolated from tissues or organs other than the bone marrow, e.g. from the muscles. The purpose is to evaluate if source-cells from these other locations are better for implantation than source-cells from the bone marrow.

Panel 10: same as Panel 2, except the “source-cells” will be isolated from tissues or organs other than the bone marrow, e.g. from the muscles. The purpose is to evaluate if source-cells from these other locations are better for implantation than source-cells from the bone marrow.

Panel 11: same as Panel 3, except the “source-cells” will be isolated from tissues or organs other than the bone marrow, e.g. from the muscles. The purpose is to evaluate if source-cells from these other locations are better for implantation than source-cells from the bone marrow.

Panel 12: same as Panel 4, except the “source-cells” will be isolated from tissues or organs other than the bone marrow, e.g. from the muscles. The purpose is to evaluate if source-cells from these other locations are better for implantation than source-cells from the bone marrow.

Panel 13: same as Panel 5, except the “source-cells” will be isolated from tissues or organs other than the bone marrow, e.g. from the muscles. The purpose is to evaluate if source-cells from these other locations are better for implantation than source-cells from the bone marrow.

Panel 14: same as Panel 6, except the “source-cells” will be isolated from tissues or organs other than the bone marrow, e.g. from the muscles. The purpose is to evaluate if source-cells from these other locations are better for implantation than source-cells from the bone marrow.

Panel 15: same as Panel 7, except the “source-cells” will be isolated from tissues or organs other than the bone marrow, e.g. from the muscles. The purpose is to evaluate if source-cells from these other locations are better for implantation than source-cells from the bone marrow.

Panel 16: same as Panel 8, except the “source-cells” will be isolated from tissues or organs other than the bone marrow, e.g. from the muscles. The purpose is to evaluate if source-cells from these other locations are better for implantation than source-cells from the bone marrow.

### Part Four: Effect of FAS on Exercise Endurance and Life Span

### Introduction:

Fibroplate Inc. has developed a product called Fibrinoplate-S (FPS) which contains the active substance called Fibrinogen coated Albumin Spheres (FAS).

FAS has been shown to mobilize stem cells from the bone marrow, which will exit the bone marrow to arrive at wound sites to differentiate into the various tissues needed to heal the wound (more CD34+ cells at the wound site; neo-vascularization more complete at the FPS-treated group than the saline-group.)

FAS administered to whole-body lethally irradiated mice resulted in 60% survival rate in the FPS group compared to 5% in the saline-group, via a mechanism of reducing bleeding internally to the brain or GI, plus mobilization of stem cells for recovery.

Purpose of this Pilot Study:

The hypothesis: the intravenous administration of FPS into old mice will increase their muscle mass leading to increased exercise endurance (and secondarily to longevity.) The data, if useful will be included in a Phase I grant application to the NIH.

### Design of the Study:

20 old mice (18 month old, C57BL/6J?) will be divided into 2 groups (10 each, 5 male, 5 female). Exercise first to produce stress; then drug/saline admin.
Mice will be administered intravenously weekly doses (4 ml per kg each: i.e. 4 ml of FPS (equal to 32 mg spheres) per kg; or 4 ml of saline per kg).
Mild exercise everyday (to keep healthy)?

### Day of Exercise Endurance Test and Treatment (Table 5):

TABLE 5

| | Day | Day | Day | Day | | End of |
|---|---|---|---|---|---|---|
| | 0 | 7 | 14 | 21 | Day 42 | life |
| Exercise | yes | yes | yes | yes | yes | One |
| till mice | | | | | | more |
| fail test | | | | | | exercise |
| | | | | | | endurance |
| | | | | | | test near |
| | | | | | | end of |
| | | | | | | life |
| Admin | yes | yes | yes | yes | No drugs | Option: |
| saline vs | | | | | or saline, | Keep 6 |
| FPS 2 hr | | | | | sacrifice | mice each |
| after test | | | | | for muscle | group (3 |
| on same | | | | | biopsy | male, 3 |
| day | | | | | study if | female) |
| | | | | | there is a | till end |
| | | | | | difference | of life? |
| | | | | | in endurance | |

### Data to Collect:

Duration of exercise tolerance.

If there is a difference between FPS-group vs saline, measure muscle mass and muscle stem cells (MuSC) concentration in biopsy (plus another other meaningful parameters.)

### Part Five: Application of Nanoparticles Including FAS for Space Experiments

A Novel Approach to Identify Mechanisms of Irradiation Damage Under Microgravity

### Introduction:

Conventional drug discoveries typically take a two-step approach: (a) identify the mechanism of harm; (b) devise a countermeasure against that mechanism to prevent, mitigate or treat the disease. However, many health problems have more than one mechanism of injury; resolution of one point-of-harm may not significantly reduce the severity of the overall problem. One example is the demonstration of the efficacy of Fibrinogen-coated Albumin Nanospheres (FAS) in improving the survival rate in total-body irradiated mice from 5% to 60% (Radiat Res. 194:162-172, 2020). The mechanism has been shown to be the control of lethal internal bleeding caused by high dose irradiation. However, long-term survival may depend on the existence of another mechanism, which was later demonstrated in an irradiation-induced skin injury model to be the mobilization of stem cells to the wound site to cause rapid wound healing (Radiat Res poster 2019). Fortunately, FAS has both functions, resulting in benefits not expected from mitigation of only one mechanism of harm. Furthermore, the mechanism of harm during space travel can be subtle and may not be detectable by conventional methos, especially in situations of low-level irradiation, with the compounding effects of microgravity. We, therefore, propose a novel approach, which is to start with the demonstration of medical benefit (including the mobilization of stem cells to replace damaged tissues) before we work backwards towards the identification of the contributing mechanisms of harm, the understanding of which will lead to a more holistic approach for long term well-being of earth-bound citizens and astronauts.

### Published Data:

Mao et al. have published several documents relating space environment to brain injury, including (a) Long-term effects of simulated microgravity and/or chronic exposure to low-dose gamma radiation on behavior and blood-brain barrier integrity. NPJ Microgravity. 2016 Jun. 9; 2:16019. (b) Simulated Microgravity and Low-Dose/Low-Dose-Rate Radiation Induces Oxidative Damage in the Mouse Brain. Radiat Res. 2016 June; 185(6):647-57. (c) Immunological and hematological outcomes following protracted low dose/low dose rate ionizing radiation and simulated microgravity. Sci Rep. 2021 Jun. 1; 11(1):11452. On the other hand, Yen has published data and obtained patents showing that FAS can be useful as a countermeasure against various radiation- or trauma-induced injuries involving soft tissues and hard tissues (bone fractures) on earth-bound conditions. See: U.S. Pat. No. 11,260,110 “Nanoparticles for the therapeutic treatment of radiation-induced skin ulcers”; U.S. Pat. No. 11,260,109 “Albumin nanoparticles to augment stem cell function in vivo”; U.S. Pat. No. 12,161,697 “Nanospheres for bone fracture”; U.S. Pat. No. 9,226,898 “Submicron particles for the treatment of radiation damage in patients”.

### Hypothesis:

All long-term damage on earth or in space is ultimately due to the failure of the replacement system (i.e. regeneration from progenitor cells of whatever site of origin) not being able to catch up with the rate of damage; and FAS can remedy that inefficiency.

### Novel Approach:

This new method will use the End Result together with the Starting Event to bracket and identify the essential mechanism of healing/repair. In separate experiments, FAS has been known to (a) bind progenitor cells in the bone marrow of healthy animals (Starting Event); (b) increase the concentration of CD34+ cells in the bone marrow; followed by (c) impact neutrophils in peripheral blood within 2 days and increase WBC (lymphocytes, monocytes, granulocytes) 51 days post-FAS dose; (d) in the presence of a wound (on skin, or bone fracture) FAS accelerates the healing process (End Result) via accelerated neo-vascularization leading to a healed wound site. This proposal will link up the above events by lineage study using Green Fluorescent Protein (GFP)-expressing stem cells isolated from genetically modified mice where the End Result resides with the healing of the brain.

In Phase I: using data already established, we will administered FAS (8 mg/kg) consistent with the protocol disclosed by Sung (Radiat Res. 2020 Aug. 1; 194(2): 162-172) (on day 5, 10, 15) with low dose irradiation and hind-limb unloading started on Day 0. Mice will be sacrificed on day 45. If no statistically significant change in the brain oxidative damage parameter or histological change, we will increase the dose to 16 mg per kg.

In Phase II: we will collect GFP-stem cells from mice purchased from Jackson Laboratory and implant them into compatible (non-GFP) mice. Using the optimal conditions found in Phase I, we will track the GFR-stem cells from the bone marrow to the brain after FAS treatment, and any hematopoietic cell populations derived from the implanted stem cells. End-point: presence of GFP-brain cells in injured brain or increased CD31+ endothelial cells (re-vascularization) in the FAS group compared to the control group will validate the hypothesis that FAS can mobilized stem cells from the bone marrow to serve as countermeasure against low-dose irradiation injury under microgravity, and re-vascularization is one mechanism of repair in an organ such as the brain.

### Summary:

Fibrinogen-coated Albumin Nanospheres (FAS) has shown that it can mobilize bone marrow cells into the blood which then go to injured soft and hard tissues to achieve accelerated repair. We hypothesize that (a) brain injuries caused by low-dose irradiation under simulated microgravity can be mitigated by the administration of FAS; (b) the mechanism is from acceleration of neo-vascularization, proven by lineage study of GFP-labeled bone marrow cells implanted into compatible non-GFP animals before injury, which regenerate into functional GFP-brain cells after FAS treatment.

According to one aspect, the present technology can include a method of using an albumin nanoparticle suspension containing submicron albumin spheres to enrich and channel pluripotent cells in a subject in need thereof. The method can include the step of administering intravenously a therapeutically effective amount of the albumin nanoparticle suspension containing the submicron albumin spheres to the subject. The albumin spheres can be configured to provide acceleration of in vivo neo-vascularization of the pluripotent cells to enrich one or more of the pluripotent cells into desirable cells for proper physiological function and to channel the one or more enriched pluripotent cells so the enriched pluripotent cells are reprogrammed to differentiate into pathways useful to the subject.

According to another aspect, the present technology can include a method of using an albumin nanoparticle suspension containing submicron albumin spheres to enrich and channel pluripotent cells in a subject in need thereof. The method can include the steps of providing a suspension including fibrinogen-coated albumin nanospheres prepared by coating blank albumin spheres with a solution containing human fibrinogen. Administering intravenously a therapeutically effective amount of the albumin nanoparticle suspension to the subject. Enriching one or more of the pluripotent cells into desirable cells for proper physiological function by in vivo neo-vascularization of the one or more pluripotent cells. Channeling the one or more enriched pluripotent cells so the enriched pluripotent cells are reprogrammed to differentiate into pathways useful to the subject.

In some embodiments, the albumin spheres of the albumin nanoparticle suspension can be bound with fibrinogen molecules to produce Fibrinogen Albumin Spheres (FAS).

Some embodiments of the present technology can include a step of increasing a concentration of CD34+ or CD31− cells by the Fibrinogen Albumin Spheres.

In some embodiments, the channeling of the enriched pluripotent cells can be configured to replenish a depletion of stem cells in bone marrow of the subject.

Some embodiments of the present technology can include the steps of:
moving of the FAS from an intravenous compartment into a bone marrow compartment of the subject;
attachment of the FAS to the pluripotent cells, and signaling to the pluripotent cells to reproduce to create FAS-attached pluripotent cells;
exiting of the FAS-attached pluripotent cells from the bone marrow;
maturation of the FAS-attached pluripotent cells in peripheral blood of the subject;
arriving of the FAS-attached pluripotent cells at a target site of the subject; and
healing macroscopically or histologically at the target site by an abundance of the FAS-attached pluripotent cells at the target site.

In some embodiments, the depletion of stem cells can be caused by ablation or partial ablation.

In some embodiments, the desired cells can be functional cells at a target tissue of the subject.

The method according to claim 1 further comprising the step of promoting by the albumin spheres a development of lineages of pluripotent cells needed at a target tissue of the subject.

In some embodiments, the lineages can be of hematopoietic cells.

In some embodiments, the pluripotent cells can be from a donor that is not the subject.

Some embodiments of the present technology can include a step of increasing an effectiveness of engrafting of the donor pluripotent cells in bone marrow of the subject resulting in an increase in survivability of the donor pluripotent cells in the bone marrow of the subject and to become mature cells later.

While embodiments of the enrichment and channeling of pluripotent cells by protein nanoparticles have been described in detail, it should be apparent that modifications and variations thereto are possible, all of which fall within the true spirit and scope of the present technology. With respect to the above description then, it is to be realized that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A method of using an albumin nanoparticle suspension containing submicron albumin spheres to enrich and channel pluripotent cells in a subject in need thereof, the method comprising the step of administering intravenously a therapeutically effective amount of the albumin nanoparticle suspension containing the submicron albumin spheres to the subject, the albumin spheres being configured to provide acceleration of in vivo neo-vascularization of the pluripotent cells to enrich one or more of the pluripotent cells into desirable cells for proper physiological function and to channel the one or more enriched pluripotent cells so the enriched pluripotent cells are reprogrammed to differentiate into pathways useful to the subject.

2. The method according to claim 1, wherein the albumin spheres of the albumin nanoparticle suspension are bound with fibrinogen molecules to produce Fibrinogen Albumin Spheres (FAS).

3. The method according to claim 2 further comprising the step of increasing a concentration of CD34+ or CD31− cells by the Fibrinogen Albumin Spheres.

4. The method according to claim 2, wherein channeling of the enriched pluripotent cells is configured to replenish a depletion of stem cells in bone marrow of the subject.

5. The method according to claim 2 further comprising the steps of:
moving of the FAS from an intravenous compartment into a bone marrow compartment of the subject;
attachment of the FAS to the pluripotent cells, and signaling to the pluripotent cells to reproduce to create FAS-attached pluripotent cells;
exiting of the FAS-attached pluripotent cells from the bone marrow;
maturation of the FAS-attached pluripotent cells in peripheral blood of the subject;
arriving of the FAS-attached pluripotent cells at a target site of the subject; and
healing macroscopically or histologically at the target site by an abundance of the FAS-attached pluripotent cells at the target site.

6. The method according to claim 4, wherein the depletion of stem cells is caused by ablation or partial ablation.

7. The method according to claim 1, wherein the desired cells are functional cells at a target tissue of the subject.

8. The method according to claim 1 further comprising the step of promoting by the albumin spheres a development of lineages of pluripotent cells needed at a target tissue of the subject.

9. The method according to claim 8, wherein the lineages is of hematopoietic cells.

10. The method according to claim 1, wherein the pluripotent cells are from a donor that is not the subject.

11. The method according to claim 10 further comprising the step of increasing an effectiveness of engrafting of the donor pluripotent cells in bone marrow of the subject resulting in an increase in survivability of the donor pluripotent cells in the bone marrow of the subject and to become mature cells later.

12. A method of using an albumin nanoparticle suspension containing submicron albumin spheres to enrich and channel pluripotent cells in a subject in need thereof, the method comprising the steps of:
a) providing a suspension including fibrinogen-coated albumin nanospheres prepared by coating blank albumin spheres with a solution containing human fibrinogen;
b) administering intravenously a therapeutically effective amount of the albumin nanoparticle suspension to the subject;
c) enriching one or more of the pluripotent cells into desirable cells for proper physiological function by in vivo neo-vascularization of the one or more pluripotent cells; and
d) channeling the one or more enriched pluripotent cells so the enriched pluripotent cells are reprogrammed to differentiate into pathways useful to the subject.

13. The method according to claim 12 further comprising the step of increasing a concentration of CD34+ or CD31− cells by the Fibrinogen Albumin Spheres.

14. The method according to claim 12, wherein channeling of the enriched pluripotent cells is configured to replenish a depletion of stem cells in bone marrow of the subject.

15. The method according to claim 12 further comprising the steps of:
moving of the FAS from an intravenous compartment into a bone marrow compartment of the subject;
attachment of the FAS to the pluripotent cells, and signaling to the pluripotent cells to reproduce to create FAS-attached pluripotent cells;
exiting of the FAS-attached pluripotent cells from the bone marrow;
maturation of the FAS-attached pluripotent cells in peripheral blood of the subject;
arriving of the FAS-attached pluripotent cells at a target site of the subject; and
healing macroscopically or histologically at the target site by an abundance of the FAS-attached pluripotent cells at the target site.

16. The method according to claim 15, wherein the depletion of stem cells is caused by ablation or partial ablation.

17. The method according to claim 12, wherein the desired cells are functional cells at a target tissue of the subject.

18. The method according to claim 12 further comprising the step of promoting by the albumin spheres a development of lineages of pluripotent cells needed at a target tissue of the subject.

19. The method according to claim 12, wherein the pluripotent cells are from a donor that is not the subject.

20. The method according to claim 19 further comprising the step of increasing an effectiveness of engrafting of the donor pluripotent cells in bone marrow of the subject resulting in an increase in survivability of the donor pluripotent cells in the bone marrow of the subject and to become mature cells later.
